(19) United States
(12) Patent Application Publication
(10) Pub. No.: US 2026/0000584 A1
(43) Pub. Date: Jan. 1, 2026

(54) PROCESS FOR MANUFACTURING A PERSONAL CARE COMPOSITION

(71) Applicant: The Procter & Gamble Company, Cincinnati, OH (US)

(72) Inventors: Olubolaji Akintelure, Mason, OH (US); Timothy John Walter Cilley, Mason, OH (US); Timothy Charles Gulbin, Maineville, OH (US); Karl Shiqing Wei, Mason, OH (US)

(21) Appl. No.: 19/251,158

(22) Filed: Jun. 26, 2025

Related U.S. Application Data
(60) Provisional application No. 63/664,775, filed on Jun. 27, 2024.

(51) Int. Cl. A61K 8/06; A61K 8/37; A61K 8/44; A61Q 19/10

(52) U.S. Cl. CPC A61K 8/06; A61K 8/375; A61K 8/44; A61Q 19/10; A61K 2800/33

(57) ABSTRACT

A process for manufacturing a personal care composition having a cleansing phase and a benefit phase. The composition is substantially free of alkyl sulfate and alkyl ether sulfate type of surfactants. The process includes the steps of: forming the cleansing phase of the personal care composition in a main mixing vessel on a batch production line, admixing the benefit phase to the cleansing phase on a flow line downstream of the batch production line; and recovering the resultant personal care composition.

### FIELD OF THE INVENTION

The present application generally relates to a process for manufacturing a personal care composition. The process leads to a personal care composition that can further provide or enhance skin moisturization and skin barrier function. The personal care composition also exhibits improved stability and provide desired rheology, viscosity and lather properties. The present application relates to personal care compositions, particularly to personal cleansing compositions for cleansing hair or skin. More specifically, a personal care composition includes a cleansing phase and a benefit phase. The personal care composition pertains to a sulfate-substantially free surfactant system. The personal care composition is substantially free of alkyl sulfate or alkyl ether sulfate type of surfactants.

### BACKGROUND OF THE INVENTION

Cleansing the skin has been a common practice for centuries, with early cleansers relying on soap chemistry or mechanical action to remove dirt, sweat, sebum, and body odors. Soap-based cleansers and body washes have been widely used, along with various personal care compositions, to achieve effective skin cleansing.

Personal care compositions often require the inclusion of structuring agents to suspend and stabilize dispersions of benefit agents while maintaining the physical integrity of the composition. The ability to provide structure is crucial for the overall performance of personal care compositions. However, striking the right balance between structure and micellar formation upon dilution is a challenging task. Excessive structure can result in inferior performance, while inadequate structure may lead to instability of the composition.

In recent years, personal care compositions containing sodium trideceth sulfate and a structuring system based on specific associative polymers have been explored. These compositions have shown promising results in terms of providing the desired structure and cleansing efficiency. However, sodium trideceth sulfate is a sulfate-based surfactant. There is a growing demand for sulfate-free alternatives due to concerns regarding potential skin irritation and environmental impact.

Therefore, there is always an interest for providing a personal care composition that eliminates the use of sodium trideceth sulfate while maintaining the desired rheology, viscosity and lather properties. Such a composition would address the demand for sulfate-free formulations while providing effective skin cleansing and deposition of benefit agents.

Maintaining optimal skin moisturization and preserving the integrity of the skin barrier are also aspects when providing personal care. Traditional sulfate-free compositions comprising benefit agents such as natural emollients have been widely used to provide hydration and skin's protective function. However, there is still a need to develop a manufacturing process for personal care compositions that can further provide or enhance skin moisturization and skin barrier function.

### SUMMARY OF THE INVENTION

A process for manufacturing a personal care composition, preferably a semi-continuous or continuous process, the process comprising a cleansing phase and a benefit phase, wherein the composition is substantially free of alkyl sulfate and alkyl ether sulfate type of surfactants is provided and comprises the steps of, preferably in that order:
(a) forming the cleansing phase of the personal care composition in a main mixing vessel on a batch production line; the cleansing phase being an aqueous structured surfactant phase; wherein the cleansing phase comprises: (a1) an anionic surfactant being substantially free of sulfate, preferably a N-acyl amino acid surfactant, most preferably a N-acyl alaninate surfactant; (a2) a zwitterionic or amphoteric surfactant; and (a3) a structuring system comprising from about 0.5 wt. % to about 5 wt. % of an emulsifying agent; and from about 0.01 wt. % to about 10 wt. % of a rheology modifier;
(b) admixing the benefit phase to the cleansing phase on a flow line downstream of the batch production line, wherein the benefit phase comprises from about 0.1 wt. % to about 50 wt. % of a benefit agent; and
(c) recovering the resultant personal care composition.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 provides a process diagram for a process for manufacturing a personal care composition; and

FIG. 2 provides two images of a respective comparative example made by conventional mixing and an example A according to the present disclosure, both as neat products under a differential interference contrast optical microscope with a 10× objective lens.

### DETAILED DESCRIPTION OF THE INVENTION

### Definitions of Terms

In this document, the following definitions apply unless specifically stated otherwise.

All percentages are by weight (w/w) of the composition, unless otherwise specified. “% wt.” means percentage by weight. References to ‘parts’ e.g. a mixture of 1 part X and 3 parts Y, is a ratio by weight. All ratios or percentages are weight ratios or weight percentages unless specifically stated otherwise.

An “active composition” is the composition absent water, and an “active ingredient” is the ingredient absent its water.

“QS” or “QSP” means sufficient quantity for 100% or for 100 g. +/− indicates the standard deviation. All ranges are inclusive and combinable. The number of significant digits conveys neither a limitation on the indicated amounts nor on the accuracy of the measurements. All numerical amounts are understood to be modified by the word “about”.

All measurements are understood to be made at 25° C. and at ambient conditions, where “ambient conditions” means at 1 atmosphere (atm) of pressure and at 65% relative humidity, unless otherwise stated. “Relative humidity” refers to the ratio (stated as a percent) of the moisture content of air compared to the saturated moisture level at the same temperature and pressure. Relative humidity can be measured with a hygrometer, in particular with a probe hygrometer from VWR® International.

Herein “min” means “minute” or “minutes”. Herein “mol” means mole. Herein “g” following a number means “gram” or “grams”. “Ex.” means “example”. All amounts as they pertain to listed ingredients are based on the active level and do not include carriers or by-products that may be included in commercially available materials.

Herein, “comprising” means that other steps and other ingredients can be in addition. “Comprising” encompasses the terms “consisting of” and “consisting essentially of”. The compositions, methods, uses, and processes described herein can comprise, consist of, and consist essentially of the elements and limitations described herein, as well as any of the additional or optional ingredients, components, steps, or limitations described herein. Embodiments and aspects described herein may comprise or be combinable with elements, features or components of other embodiments and/or aspects despite not being expressly exemplified in combination, unless an incompatibility is stated.

As used herein, the articles including “a” and “an” when used in a claim, are understood to mean “one or more” of what is claimed or described.

The terms “include,” “includes,” and “including,” as used herein are meant to be non-limiting.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the composition.

The term “free of” as used herein means that the composition comprises 0% of an ingredient by weight of the composition, thus no detectable amount of the stated ingredient.

The term “substantially free of” as used herein means less than about 1.5%, less than about 1.2%, less than about 1%, less than about 0.8%, less than about 0.5%, less than about 0.3%, less than about 0.1%, less than about 0.01% or less than an immaterial amount of by weight of the composition.

Herein “Comp. Ex.” or “C. Ex.” means comparative example; and “Ex.” means example.

The term “molecular weight” or “M.Wt.” as used herein refers to the weight average molecular weight unless otherwise stated. The weight average molecular weight can be measured by gel permeation chromatography (“GPC”).

The term “personal care composition” as used herein, refers to compositions intended for topical application to the skin, hair, or scalp. The compositions described herein are rinse-off formulations, in which the product is applied topically to the skin, hair, or scalp and then is subsequently rinsed within minutes from the skin or hair or scalp with water, or otherwise wiped off using a substrate with deposition of a portion of the composition. The compositions also may be used as shaving aids. The personal care composition is typically extrudable or dispensible from a package. The personal care compositions typically exhibit a Carreau zero shear viscosity of from about 200 Pa·s (200,000 centipoise (cP)) to about 16 000 Pa·s (16,000,000 cP); or from about 500 Pa·s (500,000 centipoise (cP)) to about 16 000 Pa·s (16,000,000 cP); or from about 500 Pa·s (500,000 centipoise (cP)) to about 13 000 Pa·s (13,000,000 cP), or from about 500 Pa·s (500,000 centipoise (cP)) to about 7 750 Pa·s (7,750,000 cP); or from about 1 500 Pa·s (1,500,000 centipoise (cP)) to about 16 000 Pa·s (16,000,000 cP) as measured by the Carreau Zero Shear Viscosity Method as disclosed herein. The personal care compositions can be in the form of liquid, semi-liquid, cream, lotion or gel compositions intended for topical application to skin. Examples of personal care compositions can include but are not limited to shampoo, conditioning shampoo, body wash, moisturizing body wash, shower gels, skin cleansers, cleansing milks, hair and body wash, in shower body moisturizer, pet shampoo, shaving preparations and cleansing compositions used in conjunction with a disposable cleansing cloth.

The term “personal cleansing composition” as used herein refers to compositions intended for topical application to the hair and the skin, preferably to the skin, for cleansing.

The term “mixtures” as used herein is meant to include a simple combination of materials and any compounds that may result from their combination.

The term “room temperature” refers to a temperature of 25° C.

The term “rinse-off” as used herein means the intended product usage includes application to skin followed by rinsing and/or wiping the product from the skin within a few seconds to minutes of the application step. The product is generally applied and rinsed in the same usage event, for example, a shower or washing one's hands.

The term “derivative” as used herein refers to structures which are not shown but which one skilled in the art would understand are variations of the basic compound.

The term “structured,” as used herein means having a rheology that confers stability on the personal care composition. The personal care composition having at least a cleansing phase and a benefit phase may be defined as a multiphase composition. The degree of structure is determined by characteristics determined by one or more of the following methods: The Carreau Zero Shear Viscosity Method or by the Ultracentrifugation Method, all in the Test Methods below. Accordingly, a cleansing phase of the personal care composition or the personal care composition is considered “structured,” if the surfactant cleansing phase or the personal care composition has one or more of the following properties described below according to the Carreau Zero Shear Viscosity Method or by the Ultracentrifugation Method. A surfactant phase is considered to be structured, if the phase has one or more of the following characteristics:
A. a Carreau Zero Shear Viscosity from about 200 Pa·s to about 16 000 Pa·s, preferably from about 500 Pa·s to about 16 000 Pa·s, or from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11600 Pa·s; or from about 500 Pa·s to about 7750 Pa·s; or from about 1 500 Pa·s to about 16 000 Pa·s as measured by the Carreau Zero Shear Viscosity Method as disclosed herein; or
B. a Structured Domain Volume Ratio as measured by the Ultracentrifugation Method described hereafter, of greater than about 40%, or at least about 50%, or at least about 55%, or at least about 60 or at least about 65%, or at least about 70%, or at least about 75%, or at least about 80%, or at least about 85%, or at least about 90%.

The term “lather” as used herein, means an aerated foam which results from providing energy to aqueous surfactant mixtures, especially dilute mixtures. Lather is increased in micellar compositions compared to structured, e.g., lamellar compositions, so that a phase change during dilution to micelles typically increases lather.

The term “visually distinct” as used herein, refers to a region of the personal care composition having one average composition, as distinct from another region having a different average composition, wherein the regions are visible to the unaided naked eye. This would not preclude the distinct regions from comprising two similar phases where one phase could comprise pigments, dyes, particles, and various optional ingredients, hence a region of a different average composition. A phase generally occupies a space or spaces having dimensions larger than the colloidal or sub-colloidal components it comprises. A phase can also be constituted or re-constituted, collected, or separated into a bulk phase in order to observe its properties, e.g., by centrifugation, filtration or the like.

The term “batch production line” as used herein, refers to the section of a production line where the ingredients are mixed with each other one batch at a time in an individual vessel or tank. In the disclosure herein, for an instance, the ingredients are added to a main tank or main mixing vessel, mixed together, and allowed to react or blend for a specific period. Additional mixing may be provided by a recirculation loop. The loop may also include a heat exchanger to control the temperature of the batch. Once the desired process is complete, the entire batch is transferred to a reservoir or the next production line that is a flow line downstream the batch production line in the disclosure herein. The process may be then repeated for the next batch. This type of production is commonly used when the process requires individual batches or when it is not feasible or economical to continuously flow the ingredients.

The term “flow line” as used herein, refers to the section of the production line where the product (i.e., a cleansing phase) of the main mixing vessel is pumped out through a pipeline, and additional ingredients (e.g., a benefit phase) are continuously added and mixed while the product flows. The phases (e.g., cleansing and benefit phases) are mixed as it continuously makes its way through a series of tanks, tubes, mixing devices, or other vessels. The resulting mixture is continuously transferred to a reservoir or the packaging filling line without interruption. This type of production is often used when a continuous and uninterrupted supply of the product is desired, or when the process is more efficient when operated continuously.

The term “semi-continuous process” as used herein, refers to a combination of a batch and continuous processes. It involves periodic or intermittent production cycles where some steps are performed continuously, while others are performed in a batch mode. For example, in a semi-continuous process, certain operations may be carried out continuously, while others, such as filling or packaging, may be done in batches.

The term “continuous process” as used herein, refers to where the cleansing phase is continuously formulated prior to it being mixed with the benefit phase. In a continuous process, the cleansing and benefit phases are mixed as it continuously makes its way through a series of tanks, tubes, pipes, mixing devices, or other vessels. In a semi-continuous process, some but not all of the mixing steps are continuous.

The processes, personal care compositions, methods and uses of the compositions, the structures and the respective compositions as described in the Summary or as described hereinbelow are for fulfilling the technical effects or goals as set out herein. These objects and other advantages as may be apparent to those skilled in the art can be achieved through the personal care compositions, methods and uses of the compositions as described herein.

### Benefits

A manufacturing process for a personal care composition is provided, and specifically designed to enhance the moisturization performance and skin barrier function compared to conventional sulfate-free compositions containing benefit agents such as natural emollients. The key advantages of the process disclosed herein are as follows:
1. Enhanced Moisturization Performance: The incorporation of benefit agents such as natural lipids using the proposed process results in the creation of larger lipid particles, which significantly improves the moisturization performance of the composition. This leads to superior skin hydration, ensuring long-lasting moisturization and improved overall skin health.
2. High Deposition of benefit agents: The manufacturing process achieves high deposition of benefit agents in sulfate-free surfactant compositions. This ensures that the beneficial ingredients (benefit agents) are effectively delivered to the skin, maximizing their efficacy and providing enhanced moisturization and skin barrier support.
3. Uniform Temperature Control: The process carefully controls the temperature just above the melting point of the benefit agent during dispersion into the cleansing phase and emulsification in the respective mixing device. This ensures the formation of larger particles for the benefit agents, maximizing their effectiveness and ensuring uniform distribution throughout the composition.
4. Advanced Bioavailability and Penetration: The manufacturing process incorporates advanced techniques, such as emulsion stabilization methods, and targeted delivery systems. These techniques enhance the bioavailability and penetration of benefits agents such as natural emollients into the skin, allowing for maximum hydration and strengthening of the skin barrier.
5. Long-Term Stability: The manufacturing process addresses the challenge of long-term stability by employing formulation strategies that minimize product degradation, phase separation, and undesirable changes in appearance. This ensures that the composition maintains its efficacy and aesthetic appeal over its shelf life.

In summary, the manufacturing process described herein offers significant consumer benefits, including improved skin moisturization, enhanced skin barrier function, and superior overall skin health. By optimizing formulation parameters and incorporating advanced techniques, this process allows for the creation of a sulfate-free personal care composition that surpasses the performance of conventional compositions containing benefit agents.

The personal care composition made by the process comprises a sulfate-free surfactant system, which provides improved rheology, viscosity and lather properties compared to conventional sulfate-based systems.

By eliminating sodium trideceth sulfate, the personal care composition reduces the risk of skin irritation and environmental impact associated with sulfate-based surfactants. Instead, the composition includes a N-acyl alaninate surfactant, in combination with a specific structuring system combining an emulsifying agent with a rheology modifier as defined herein. Such personal care composition can achieve the desired structure necessary for suspending and stabilizing benefit agents.

The resulting personal care composition exhibits excellent cleansing efficacy, effectively removing dirt, sweat, sebum, and body odors from the skin. Additionally, it ensures the rapid formation of micelles upon dilution, facilitating the deposition of benefit agents onto the skin.

The personal care composition offers an improved rheology or viscosity, providing enhanced texture, spreadability, and foam generation. This leads to a luxurious sensory experience during use, enhancing consumer satisfaction and acceptance of the product.

### Process for Manufacturing a Personal Care Composition

A process for manufacturing a personal care composition is provided and comprises a cleansing phase and a benefit phase. Preferably, the process may be a semi-continuous or continuous process. The composition is substantially free of alkyl sulfate and alkyl ether sulfate type of surfactants. The process comprises the steps of, preferably in that order:
(a) forming the cleansing phase of the personal care composition in a main mixing vessel on a batch production line; the cleansing phase being an aqueous structured surfactant phase; wherein the cleansing phase comprises:
   (a1) an anionic surfactant being substantially free of sulfate, preferably a N-acyl amino acid surfactant, most preferably a N-acyl alaninate surfactant;
   (a2) a zwitterionic or amphoteric surfactant; and
   (a3) a structuring system comprising:
      (i) from about 0.5 wt. % to about 5 wt. % of an emulsifying agent; and
      (ii) from about 0.01 wt. % to about 10 wt. % of a rheology modifier;
(b) admixing the benefit phase to the cleansing phase on a flow line downstream of the batch production line, wherein the benefit phase comprises from about 0.1 wt. % to about 50 wt. % of a benefit agent; and
(c) recovering the resultant personal care composition.

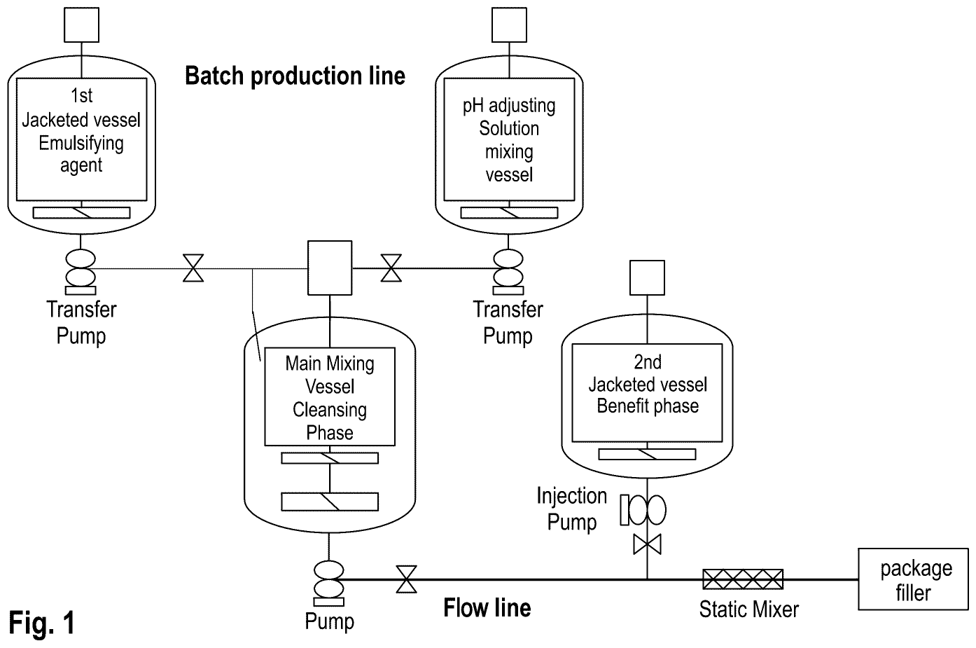

A sample process diagram for a manufacturing method is shown in FIG. 1.

The personal care composition pertains to a sulfate-substantially free surfactant system. In other words, the personal care composition is substantially free of alkyl sulfate and/or alkyl ether sulfate type of surfactant. Namely, the personal care composition comprises less than about 1.5%, or less than about 1.2%, or less than about 1%, or less than about 0.8%, or less than about 0.5%, or less than about 0.3%, or less than about 0.1%, or less than about 0.01% or is free of alkyl sulfate and/or alkyl ether sulfate type of surfactant by weight of the composition.

Preferably, the personal care composition may comprise less than about 1.5%, or less than about 1.2%, or less than about 1%, or less than about 0.8%, or less than about 0.5%, or less than about 0.3%, or less than about 0.1%, or less than about 0.01%, or is free of any alkyl sulfate which comprises C₁₂-C₁₈ alkyl sulfate and/or any alkyl ether sulfate including alkyl glyceryl ether sulfates.

More preferably, the personal care composition may comprise less than about 1.5%, or less than about 1.2%, or less than about 1%, or less than about 0.8%, or less than about 0.5%, or less than 0.3%, or less than about 0.1%, or less than about 0.01%, or is free of sodium lauryl sulfate.

Alternatively, the personal care composition may be free of alkyl sulfate and/or alkyl ether sulfate type of surfactant. Namely, the personal care composition may comprise 0% of alkyl sulfate and/or alkyl ether sulfate type of surfactant by weight of the composition, thus no detectable amount of alkyl sulfate and/or alkyl ether sulfate type of surfactant.

In that respect, the personal care composition may not comprise any alkyl sulfate which comprises C₁₂-C₁₈ alkyl sulfate and/or any alkyl ether sulfate including alkyl glyceryl ether sulfates.

The personal care composition may not comprise any alkyl ether sulfates which are those having the formula:

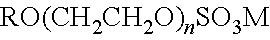

wherein R is an alkyl or alkenyl having 8 to 18 carbons, preferably 12 to 18 carbons, n has an average value of greater than at least 0.5, preferably between 2 and 3; and M is a solubilizing cation such as sodium, potassium, ammonium or substituted ammonium.

The personal care composition may not comprise any ammonium and sodium lauryl ether sulfates.

If the personal care composition does contain alkyl sulfate and/or alkyl ether sulfate type of surfactant, its content of such a weight proportion of alkyl sulfates or alkyl ether sulfate type surfactant may be less than or equal to the sum of 0.6, more preferably less than or equal to the sum of 0.2, even more preferably equal to 0.

The personal care composition may be free of any alkoxylated, preferably ethoxylated anionic surfactant.

The personal care composition may not comprise any structuring anionic surfactant selecting from the group consisting of sodium trideceth (n) sulfate (STnS) wherein n is between 0 and 3, sodium laureth (n) sulfate) wherein n is between 0 and 3, sodium tridecyl sulfate, sodium C₁₂₋₁₃ alkyl sulfate, sodium C₁₂₋₁₅ alkyl sulfate, sodium C₁₁₋₁₅ alkyl sulfate, sodium C₁₂₋₁₈ alkyl sulfate, sodium C₁₀₋₁₆ alkyl sulfate, sodium C₁₂₋₁₃ pareth sulfate, sodium C₁₂₋₁₃ pareth-n sulfate, sodium C₁₂₋₁₄ pareth-n sulfate, and mixtures thereof.

### Forming the Cleansing Phase

The process for manufacturing a personal care composition comprises the step of forming the cleansing phase of the personal care composition in a main mixing vessel on a batch production line; wherein the cleansing phase is defined as an aqueous structured surfactant phase.

The personal care composition comprises a cleansing phase. The cleansing phase comprises an anionic surfactant being substantially free of sulfate, preferably a N-acyl amino acid surfactant, most preferably a N-acyl alaninate surfactant; a zwitterionic or amphoteric surfactant; and a structuring system.

The batch production line is the section of a production line where the ingredients of the cleansing phase are mixed with each other one batch at a time in an individual vessel. The ingredients of the cleansing phase are added to a main mixing vessel, mixed together, and allowed to react or blend for a specific period.

The batch production line may be seen as a process section that involves a sequence of steps followed in a specific order in a main mixing vessel or tank.

Additional mixing may be provided by a recirculation loop. The loop may also include a heat exchanger to control the temperature of the batch. Once the desired process is complete, the entire batch is transferred to a reservoir or the next production line that is a flow line downstream the batch production line.

The batch production line has as advantages to be simple and flexible. The batch production line can be adapted; from basic, manual operations, to more sophisticated, highly automated operations. The batch production line can manage a wide range of formulae, orders of addition, and operating conditions at each step.

### Main Mixing Vessel

The cleansing phase may be prepared through conventional mixing techniques. In the present disclosure, the cleansing phase may be formed in a main mixing vessel including a turbine agitation.

In the present disclosure, the cleansing phase may be preferably formed in a main mixing vessel including side sweep and turbine agitations.

The main mixing vessel is a type of equipment used for forming the cleansing phase. It consists of a large container designed to hold the ingredients of the cleansing phase and facilitate their thorough mixing.

The main mixing vessel is the primary container or tank in the batch production line where the cleansing phase is prepared. The main mixing vessel may be typically cylindrical in shape and made of stainless steel or other suitable materials.

The main mixing vessel may be equipped with various agitators for efficient mixing. In that aspect, the main mixing vessel may include a turbine agitator, and optionally a side sweep agitator.

The main mixing vessel may be preferably jacketed for ensuring a temperature control of the cleansing phase.

The main mixing vessel may include side sweep mixing on the sides and bottom of the vessel.

In that aspect, the main mixing vessel may have no baffles.

The main mixing vessel may include a turbine agitation, and an off-center pitch blade turbine. The pitch blade turbine, which is a type of mixing impeller or agitator is commonly used in industrial mixing applications. The pitch blade turbine is a flat, disc-like impeller with several radial blades or vanes that are pitched or angled. The blades are typically curved or twisted to create a pumping and shearing action within the mixing vessel. The pitch blade turbine can help to promote efficient mixing by generating both axial and radial flow patterns.

The off-center pitch blade turbine can be defined as having the pitch blade turbine being positioned off-center within the main mixing vessel. The off-center placement of the pitch blade turbine can help to create additional flow patterns and enhance mixing efficiency, especially when combined with the side sweep mixing on the sides and bottom of the vessel or tank.

The pitch blade turbine may include at least two blades, preferably only two blades.

The turbine agitator is a type of agitator that is mounted from the top of the vessel. It consists of a set of blades or impellers that rotate to create fluid movement. The purpose of the turbine agitator is to provide additional mixing and agitation within the bulk of the composition.

The speed of the turbine agitator can vary within the specified range, typically between 10 to 90 rpm.

The side sweep agitator is a type of agitator that is mounted on the side of the vessel. It consists of a rotating arms with blades or paddles attached to it, preferably wherein the rotating arms are U-shaped. The purpose of the side sweep agitator is to scrape the sides of the vessel, ensuring that the ingredients are evenly distributed and preventing them from sticking to the walls. Also, the side sweep agitator can help for folding the ingredients of the cleansing phase in the middle of the main mixing vessel to allow the turbine uniformly mixing the ingredients.

The speed of the side sweep agitator can vary within the specified range, typically between 5 to 20 revolutions per minute (rpm).

Alternative agitators can be provided to the main mixing vessel. In one aspect, the main mixing vessel may include baffles. Instead of a side sweep agitator, the main mixing vessel may be equipped with baffles. Baffles are vertical plates or barriers that are strategically placed inside the main mixing vessel to redirect the flow of the liquid and enhance mixing of the cleansing phase. As the liquid flows around the baffles, it can create turbulence and improves the mixing efficiency. Baffles may be fixed or removable, depending on the main mixing vessel design.

In an alternate aspect, the main mixing vessel may include an anchor agitator. The anchor agitator consists of a central vertical shaft with multiple curved blades that sweep the bottom of the main mixing vessel. The anchor agitator is particularly effective for viscous or shear-sensitive cleansing phases as it provides gentle and thorough mixing. The speed of the anchor agitator may be adjusted based on the specific requirements of the cleansing phase.

In an alternate aspect, the main mixing vessel may include a paddle agitator. The paddle agitator refers to multiple flat paddles or blades that are attached to a central shaft. The paddle agitator rotates to create mixing and agitation by moving the liquid of the cleansing phase in a circular or figure-eight motion. The design of the paddles may vary, with options for angled, pitched, or radial blades depending on the desired mixing effect.

In an alternate aspect, the main mixing vessel may include a high-shear mixer: In some cases, the main mixing vessel may be paired with a high-shear mixer, especially when the addition of certain ingredients of the cleansing phase may require intense mixing or emulsification. A high-shear mixer typically consists of a rotor and a stator that create a strong shear force to break down particles and promote mixing. These mixers are also commonly used for emulsions, suspensions, and dispersion of solids in liquids.

In that disclosure, the cleansing phase may be formed in the main mixing vessel including a turbine agitation, wherein the main mixing vessel includes a turbine agitator, wherein the turbine agitator rotates at a turbine speed ranging from about 10 to about 90 rpm, or from about 10 to about 20 rpm, or from about 15 to about 25 rpm, or from about 35 to about 45 rpm, or from about 70 to about 90 rpm.

Preferably, the cleansing phase may be formed in the main mixing vessel including side sweep and turbine agitation, wherein the main mixing vessel includes a side sweep agitator, wherein the side sweep agitator rotates at a side weep speed ranging from about 5 to about 20 revolutions per minute, or from about 5 to about 10 rpm, or from about 8 to about 12 rpm, or from about 10 to about 14 rpm; and wherein the main mixing vessel includes a turbine agitator, wherein the turbine agitator rotates at a turbine speed ranging from about 10 to about 90 rpm, or from about 10 to about 20 rpm, or from about 15 to about 25 rpm, or from about 35 to about 45 rpm, or from about 70 to about 90 rpm.

More preferably, the cleansing phase may be formed in the main mixing vessel including side sweep and turbine agitation with a counter-current mixing, wherein the side sweep agitation spins in an opposition direction that the turbine agitation. Such counter-current mixing in the main mixing vessel can help achieve optimal mixing of the ingredients of the cleansing phase.

The step of forming the cleansing phase may comprise the following steps, preferably in that order:
adding water;
adding the rheology modifier under a high shear or a turbine agitation;
adding the zwitterionic or amphoteric surfactant;
optionally adding a cationic deposition polymer;
adding the anionic surfactant being substantially free of sulfate, preferably the N-acyl amino acid surfactant, most preferably the N-acyl alaninates surfactant;
adding the emulsifying agent directly to the main mixing vessel or from a first jacketed vessel to the main mixing vessel, preferably wherein the first jacketed vessel is a jacketed scrape wall vessel or a jacketed vessel including a propeller turbine equipped with a heating or cooling jacket;
optionally adding a preservative; and
adjusting the pH such that the personal care composition has a pH of from about 4.0 to about 5.5.

The step of forming the cleansing phase may comprise the additional following step: adding a perfume.

Alternatively, the step of forming the cleansing phase may comprise the following steps, preferably in that order:
adding water;
adding the zwitterionic or amphoteric surfactant;
optionally adding a cationic deposition polymer;
optionally adding a preservative;
adding the emulsifying agent directly to the main mixing vessel or from a first jacketed vessel to the main mixing vessel, preferably wherein the first jacketed vessel is a jacketed scrape wall vessel or a jacketed vessel including a propeller turbine equipped with a heating or cooling jacket;
adding the rheology modifier under a high shear or a turbine agitation;
adding the anionic surfactant being substantially free of sulfate, preferably the N-acyl amino acid surfactant, most preferably the N-acyl alaninate surfactant; and
adjusting the pH such that the personal care composition has a pH of from about 4.0 to about 5.5.

The step of forming the cleansing phase may comprise the additional following step: adding a perfume.

Each step of forming the cleansing phase together with each ingredient of the cleansing phase will be described hereinbelow.

### Water

The cleansing phase of the personal care composition may comprise water. The cleansing phase of the personal care composition may comprise from about 10% to about 90%, alternatively from about 40% to about 85%, alternatively from about 60% to about 80% by weight of water.

The step of forming the cleansing phase may comprise the following step of adding water in the main mixing vessel wherein the side sweep agitator rotates at a side weep speed ranging from about 5 to about 20 revolutions per minute, or from about 5 to about 12 rpm, or from about 5 to about 10 rpm; and wherein the main mixing vessel includes a turbine agitator, wherein the turbine agitator rotates at a turbine speed ranging from about 10 to about 25 rpm, or from about 10 to about 20 rpm, or from about 12 to about 20 rpm.

### Structuring System

The personal care composition and the cleansing phase includes a structuring system. The structuring system can help to provide structure to the cleansing phase and stability to the personal care composition.

The structuring system can help in maintaining the benefit agent, e.g. shea butter or other natural emollients, in suspension within the cleansing phase due to the presence of a plurality of lamellar vesicles formed during the said process.

The structuring system can also help in depositing the benefit agent onto skin.

The structuring system includes from about 0.5% to about 5% of an emulsifying agent by weight of the personal care composition. The structuring system further includes from about 0.01% to about 10% of a rheology modifier by weight of the personal care composition.

After adding water, the step of forming the cleansing phase may comprise the following step of adding the rheology modifier under a high shear or a turbine agitation.

Alternatively, after adding the zwitterionic or amphoteric surfactant, and optionally the cationic deposition polymer and the preservative, the step of forming the cleansing phase may comprise the following steps, preferably in that order:
adding the emulsifying agent directly or from a first jacketed vessel to the main mixing vessel;
adding the rheology modifier under a high shear or a turbine agitation.

### Rheology Modifier

After adding water in a first aspect; or alternatively the zwitterionic or amphoteric surfactant, and optionally the cationic deposition polymer and the preservative, and the emulsifying agent in a second aspect; the step of forming the cleansing phase may comprise the following step of adding the rheology modifier under a high shear or a turbine agitation.

The rheology modifier may be added to the main mixing vessel at a high shear at a turbine speed from about 30 to about 50 rpm, or from about 35 to about 50 rpm, or from about 35 to about 45 rpm, and preferably at a side sweep speed from about 10 to about 20 rpm, or from about 10 to about 17 rpm, or from about 10 to about 14 rpm. In that aspect, the side sweep agitation may be concurrent to the turbine agitation. This aspect can help optimize the relatively high shear.

Alternatively, the rheology modifier may be added to the main mixing vessel at a turbine agitation at a turbine speed from about 30 to about 50 rpm, or from about 35 to about 50 rpm, or from about 35 to about 45 rpm.

For this aspect, the main mixing vessel may include a pitch blade turbine, preferably an off-center pitch blade turbine.

As set out above, the pitch blade turbine, which is a type of mixing impeller or agitator is commonly used in industrial mixing applications. The pitch blade turbine is a flat, disc-like impeller with several radial blades or vanes that are pitched or angled. The blades are typically curved or twisted to create a pumping and shearing action within the mixing vessel. The pitch blade turbine can help to promote efficient mixing by generating both axial and radial flow patterns.

The off-center pitch blade turbine can be defined as having the pitch blade turbine being positioned off-center within the main mixing vessel. The off-center placement of the pitch blade turbine can help to create additional flow patterns and enhance mixing efficiency, especially when combined with the side sweep mixing on the sides and bottom of the vessel or tank.

The pitch blade turbine may include at least two blades, preferably only two blades.

The pitch blade turbine of the main mixing vessel can help to provide the shear used to add the rheology modifier e.g. but not limited to hydroxypropyl starch phosphate, distarch phosphate, sodium carboxymethyl starch, into the cleansing phase and to get it wetted.

Alternatively, the main mixing vessel may include a high-shear mixer: In some cases, the main mixing vessel may be paired with a high-shear mixer, especially when the addition of certain ingredients of the cleansing phase may require intense mixing or emulsification. A high-shear mixer typically consists of a rotor and a stator that create a strong shear force to break down particles and promote mixing. These mixers are also commonly used for emulsions, suspensions, and dispersion of solids in liquids.

A high-shear mixer may be used for adding the rheology modifier under a high shear. This may be the case when the rheology modifier is a powder that is difficult to be wetted, or that needs to be incorporated and dispersed or hydrated in the cleansing phase. Other apparatus may be implemented like a high shear homogenizer, or a direct injection rotor-stator homogenizer. An example of a high shear homogenizer may be Quadro® HV high shear mixer homogenizer.

Such apparatus allows dispersing the rheology modifier through a liquid stream, i.e., an aqueous phase, and providing intense shear to hydrate the rheology modifier in a powder form.

Other high shear homogenizers useful herein include, for example: Sonolator® available from Sonic Corporation, Manton Gaulin type homogenizer available from the APV Manton Corporation, the Microfluidizer available from Microfluidics Corporation, Becomix® available from A. Berents GmbH & Co.

The personal care composition comprises from about 0.01 wt. % to about 10 wt. % of the rheology modifier, preferably from about 0.1 wt. % to about 5 wt. % of the rheology modifier, more preferably from about 0.5 wt. % to about 2 wt. % of the rheology modifier, even more preferably from about 0.6 wt. % to about 1.5 wt. %, most preferably from about 0.75 wt. % to about 0.90 wt. % of the rheology modifier.

The rheology modifier may be an associative polymer. Associative polymers are polymers constituted by a hydrophilic main chain and hydrophobic side chains. Their behavior in solution is a result of competition between the hydrophobic and hydrophilic properties of their structure. The hydrophobic units tend to form aggregates constituting linkage points between the macromolecular chains. From a rheological viewpoint, associative water-soluble polymers have a very high viscosifying power in water and retain their viscosity well in a saline medium. In mixed polymer and surfactant systems, surfactant aggregates can form, which are stabilized by diverse types of interactions: electrostatic interactions, dipolar interactions, or hydrogen bonds. Associative water-soluble polymers can interact more specifically with surfactants due to their hydrophobic portions.

The hydrophilic main chain of these associative polymers can, in particular, result from polymerization of a hydrophilic monomer containing functions onto which hydrophobic chains can subsequently be grafted, for example acid functions. This method of preparing associative polymers is described in particular in the “Water Soluble Polymers”, ACS Symposium Series 467, ed. Shalaby W Shalaby et al., Am. Chem. Soc. Washington (1991), pp. 82-200. However, a water-soluble polymer of natural origin, or a natural polymer rendered water-soluble by chemical modification, can also be used. Associative polymers can also be formed by copolymerization of hydrophilic monomers and hydrophobic monomers. These hydrophobic polymers, introduced into the reaction medium in a much smaller quantity than the hydrophilic polymers, generally comprise a fatty hydrocarbon chain. This method of preparation is described in the publication by S. Biggs et. al., J. Phys Chem. (1992, 96. pp 1505-11). Rheology modifiers are substances that are added to the personal care compositions, to modify their flow properties and rheological behavior. Rheology modifiers can alter viscosity, thicken the material, or change its flow characteristics.

The rheology modifier may be selected from the group consisting of a polyacrylate, a polysaccharide, a modified polyol, an hydrophobically modified polyacrylate, an hydrophobically modified polysaccharide, and mixtures thereof.

Specifically, the rheology modifier may be selected from the group consisting of sodium polyacrylate, acrylates copolymer, Acrylates/Vinyl Isodecanoate Crosspolymer, Acrylates/C10-30 Alkyl Acrylate Crosspolymer, Acrylates/C10-30 alkyl acrylate crosspolymer including stearyl side chains with less than about 1% Hydrophobic modification, Acrylates/C10-30 alkyl acrylate crosspolymer including octyl side chains with less than about 5% Hydrophobic modification, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, PEG-150/Decyl Alcohol/SMDI Copolymer, PEG-150/stearyl alcohol/SMDI copolymer, hydroxypropyl starch phosphate, distarch phosphate, sodium carboxymethyl starch, starch, Tapioca starch, xanthan gum, gellan gum, carboxymethyl cellulose, carboxymethyl hydroxyethyl cellulose, hydroxypropyl methyl cellulose, sodium alginate, and mixtures thereof.

More specifically, the rheology modifier may be selected from the group consisting of sodium polyacrylate, acrylates copolymer, Acrylates/Vinyl Isodecanoate Crosspolymer, Acrylates/C10-30 Alkyl Acrylate Crosspolymer, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, hydroxypropyl starch phosphate, distarch phosphate, sodium carboxymethyl starch, Tapioca starch, xanthan gum, gellan gum, and mixtures thereof.

Non-limiting examples of associative polymers being a polyacrylate or an hydrophobically modified polyacrylate include sodium polyacrylate, acrylates copolymer, Acrylates/Vinyl Isodecanoate Crosspolymer (Stabylen 30 from 3V), Acrylates/C10-30 Alkyl Acrylate Crosspolymer (Pemulen TR1 and TR2), Aqupec SER-300 made by Sumitomo Seika of Japan, which is Acrylates/C10-30 alkyl acrylate crosspolymer comprising stearyl side chains with less than about 1% HM, Ammonium Acryloyldimethyltaurate/Beheneth-25 Methacrylate Crosspolymer (Aristoflex HMB from Clariant), Acrylates/Beheneth-25 Methacrylate Copolymer (Aculyn 28 from Rohm and Haas); Acrylates/Steareth-20 Methacrylate Copolymer (Aculyn 22 from Rohm and Haas), and Acrylates/Steareth-20 Methacrylate Crosspolymer (Aculyn 88 from Rohm and Haas).

Acrylate copolymers are defined as polymers of two or more monomers consisting of acrylic acid, methacrylic acid (q.v.) or one of their simple esters. Simple esters of methacrylic acid are made with simple alkyl groups such as methyl, ethyl, propyl and butyl and their respective regioisomers. An example of acrylate copolymers may be Luvimer 100 from BASF which is made of a terpolymer of tert-butyl acrylate, ethyl acrylate and methacrylic acid.

Non-limiting examples of associative polymers being a modified polyol include PEG-150/Decyl Alcohol/SMDI Copolymer (Aculyn 44 from Dow Chemical Company), and PEG-150/stearyl alcohol/SMDI copolymer (Aculyn 46 from Dow Chemical Company).

“SMDI” as used herein means saturated methylene diphenyl diisocyanate. “PEG-150/decyl alcohol/SMDI copolymer” is a copolymer of PEG-150 (q.v.), Decyl Alcohol (q.v.), and Saturated Methylene Diphenyl Diisocyanate (q.v.) (SMDI) monomers. “PEG-150/stearyl alcohol/SMDI copolymer” is a copolymer of PEG-150 (q.v.), Saturated Methylene Diphenyl Diisocyanate (q.v.) (SMDI), and Stearyl Alcohol (q.v.) monomers.

Preferably, the rheology modifier may comprise acrylates/C10-30 alkyl acrylate crosspolymer. Acrylates/C10-30 alkyl acrylate Crosspolymer is a copolymer of C10-30 alkyl acrylate and one or more monomers of acrylic acid, methacrylic acid or one of their simple esters crosslinked with an allyl ether of sucrose or an allyl ether of pentaerythritol.

An exemplary preferred acrylates/C10-30 alkyl acrylate crosspolymer may be Aqupec SER-300 made by Sumitomo Seika of Japan, which is Acrylates/C10-30 alkyl acrylate crosspolymer comprising stearyl side chains with less than about 1% Hydrophobic modification (HM). Other preferred rheology modifiers in that category may comprise stearyl, octyl, decyl and lauryl side chains.

Preferred acrylates/C10-30 alkyl acrylate crosspolymer may be Aqupec SER-150 that is acrylates/C10-30 alkyl acrylates crosspolymer comprising about C18 (stearyl) side chains and about 0.4% HM, and Aqupec HV-701EDR that is acrylates/C10-30 alkyl acrylates crosspolymer which comprises about C8 (octyl) side chains and about 3.5% HM.

The crosslinked rheology modifier may include a percentage hydrophobic modification, which is the mole percentage of monomers expressed as a percentage of the total number of all monomers in the polymer backbone, including both acidic and other non-acidic monomers. The percentage hydrophobic modification of the polymer, hereafter % HM, can be determined by the ratio of monomers added during synthesis, or by analytical techniques such as proton nuclear magnetic resonance (NMR). The alkyl side chain length can be determined similarly.

The structuring system of the cleansing phase comprises from about 0.01% to about 5%, preferably from about 0.01% to about 1%, more preferably from about 0.02% to about 0.3%, most preferably from about 0.03% to about 0.1% by weight of the personal care composition, of acrylates/C10-30 alkyl acrylate crosspolymer.

Non-limiting example of an associative polymer being a polysaccharide, or a modified polysaccharide includes starch, Tapioca starch, xanthan gum, gellan gum, carboxymethyl cellulose, carboxymethyl hydroxyethyl cellulose, hydroxypropyl methyl cellulose, sodium alginate, and mixtures thereof.

The rheology modifier may comprise xanthan gum. Xanthan gum can help to improve the stability of the personal care composition.

The structuring system of the cleansing phase may comprise from about 0.01% to about 10%, preferably from about 0.1% to about 5%, more preferably from about 0.2% to about 1%, most preferably from about 0.25% to about 0.30% by weight of the personal care composition, of xanthan gum.

Alternatively or in addition to, the rheology modifier may comprise a hydrophobically modified polysaccharide, especially a modified starch. The modified starch may be selected from the group consisting of hydroxypropyl starch phosphate, distarch phosphate, sodium carboxymethyl starch, and mixtures thereof.

In particular, the modified starch may comprise hydroxypropyl starch phosphate. Hydroxypropyl starch phosphate may be provided as Structure® XL from Nouryon, or C*HiForm™ A12747 from Cargill. Distarch phosphate may be provided as Agenajel 20.306 from Agrana Stärke. Sodium carboxymethyl starch may be provided as Vivastar® CS Instant Powder from J. Rettenmaier & Söhne.

Starch is a carbohydrate polymer consisting of a large number of glucose units linked together primarily by alpha 1-4 glucosidic bonds. The starch polymers come in two forms: linear (amylose) and branched through alpha 1-6 glucosidic bonds (amylopectin), with each glucose unit possessing a maximum of three hydroxyls that can undergo chemical substitution.

Hydroxypropyl starch phosphate is a modified starch. It is obtained in accordance with good manufacturing practice by esterification of food starch with sodium trimetaphosphate or phosphorus oxychloride combined with etherification by propylene oxide. Hydroxypropylation results in substitution of hydroxyl groups with 2-hydroxypropyl ether. In cases of cross-linking, where phosphorus oxychloride, connects two chains, the structure can be represented by: Starch-O—R—O-Starch, where R=cross-linking group and Starch refers to the linear and/or branched structure.

As a preferred aspect, the rheology modifier may comprise an hydrophobically modified polysaccharide being a modified starch. The modified starch may comprise hydroxypropyl starch phosphate.

In that aspect, the personal care composition may comprise from about 0.01 wt. % to about 10 wt. % of hydroxypropyl starch phosphate, preferably from about 0.1 wt. % to about 5 wt. % of hydroxypropyl starch phosphate, more preferably from about 0.5 wt. % to about 1.5 wt. % of hydroxypropyl starch phosphate, most preferably from about 0.6 wt. % to about 1.0 wt. % of hydroxypropyl starch phosphate.

Any rheology modifier, like a modified starch and in particular hydroxypropyl starch phosphate may be added to main mixing vessel under the high shear or the turbine agitation. The high shear or the turbine agitation with the pitch blade turbine, preferably the off-center pitch blade turbine described herein can help the rheology modifier as recited hereinbefore, or the modified starch and in particular hydroxypropyl starch phosphate proper hydration in the cleansing phase.

Alternatively as explained more in detailed below with the section about the emulsifying agent, the rheology modifier s may be in a powder form, e.g. xanthan gum. Xanthan gum may be added using a high shear homogenizer such as Quadro® HV high shear mixer homogenizer.

Alternatively, the rheology modifier such as xanthan gum may be added in the first jacketed vessel together with the emulsifying agent, e.g. glyceryl caprylate/caprate. In the first jacketed vessel, the rheology modifier and the emulsifying agent may form a slurry to be added together to the main mixing vessel.

Such rheology modifiers can help to provide significant enhancement of structure to the cleansing phase and thus the personal care composition, especially when the personal care composition comprises reduced levels of emulsifying agents; and provide said structure at relatively low levels of rheology modifiers. Also, lather can be further improved.

Another aspect may be related to the combination of two rheology modifiers. In that respect, the personal care composition may comprise a mixture of hydroxypropyl starch phosphate and xanthan gum.

The personal care composition may comprise from about 0.3 wt. % to about 1.5 wt. % of hydroxypropyl starch phosphate and from about 0.1 wt. % to about 0.5 wt. % of xanthan gum, preferably from about 0.3 wt. % to about 1.0 wt. % of hydroxypropyl starch phosphate and from about 0.1 wt. % to about 0.4 wt. % of xanthan gum, more preferably from about 0.75 wt. % to about 0.90 wt. % of hydroxypropyl starch phosphate and from about 0.2 wt. % to about 0.3 wt. % of xanthan gum.

In that aspect hydroxypropyl starch phosphate may be added to the main mixing vessel under a high shear or a turbine agitation. Xanthan gum may be added as a slurry with the emulsifying agent in the first jacketed vessel, preferably wherein the emulsifying agent comprises glyceryl caprylate/caprate; or xanthan gum may be added using a high shear homogenizer such as Quadro® HV high shear mixer homogenizer.

The composition can achieve the desired balance between improved structure and improved lather and resulting in effective skin cleansing and deposition of benefit agents.

The personal care composition may comprise a lather volume from about 375 mL to about 575 mL, preferably from about 395 mL to about 560 mL, more preferably from about 400 mL to about 545 mL, most preferably from about 450 mL to about 540 mL as measured according to the Cylinder Method as disclosed herein.

### Zwitterionic or Amphoteric Surfactant

The resulting personal care composition or the cleansing phase comprises a zwitterionic or amphoteric surfactant. After adding the rheology modifier; or just after adding water, the step of forming the cleansing phase may comprise the following step of adding the zwitterionic or amphoteric surfactant.

Suitable amphoteric or zwitterionic surfactants can include those described in U.S. Pat. Nos. 5,104,646 and 5,106,609, each of which is incorporated herein by reference.

The personal care composition or the cleansing phase comprises a zwitterionic surfactant.

The personal care composition may comprise from about 0.01 wt. % to about 20 wt. % of the zwitterionic surfactant; preferably from about 0.1 wt. % to about 10 wt. % of the zwitterionic surfactant; more preferably from about 1 wt. % to about 10 wt. % of the zwitterionic surfactant; most preferably from about 2 wt. % to about 5 wt. % of the zwitterionic surfactant.

The zwitterionic surfactant may comprise a betaine. As a preferred aspect, the zwitterionic surfactant may comprise an alkyl betaine or an alkyl amidopropyl betaine.

Examples of betaine zwitterionic surfactants may include coco dimethyl carboxymethyl betaine, cocoamidopropyl betaine (CAPB), coco-betaine, lauryl amidopropyl betaine (LAPB), oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof.

Examples of sulfobetaines may include coco dimethyl sulfopropyl betaine, stearyl dimethyl sulfopropyl betaine, lauryl dimethyl sulfoethyl betaine, lauryl bis-(2-hydroxyethyl) sulfopropyl betaine and mixtures thereof.

The zwitterionic surfactant may be selected from the group consisting of cocamidopropyl betaine, coco-betaine, lauramidopropyl betaine, and mixtures thereof.

Most preferably, the zwitterionic surfactant may comprise cocamidopropyl betaine. In that aspect, the personal care composition may comprise from about 0.01 wt. % to about 20 wt. % of cocamidopropyl betaine; preferably from about 0.1 wt. % to about 10 wt. % of cocamidopropyl betaine; more preferably from about 1 wt. % to about 10 wt. % of cocamidopropyl betaine; most preferably from about 2 wt. % to about 5 wt. % of cocamidopropyl betaine.

The step of forming the cleansing phase may comprise the following step of adding the zwitterionic surfactant dispersed in water, wherein the cleansing phase comprises from about 0.01% to about 20% of cocamidopropyl betaine; preferably from about 0.1% to about 10% of cocamidopropyl betaine; more preferably from about 1% to about 10% of cocamidopropyl betaine; most preferably from about 2% to about 5% of cocamidopropyl betaine by weight of the personal care composition.

Cocamidopropyl betaine can be sourced from BASF as Dehyton® PK 45 having a sodium chloride content between about 5.80-7.30 wt. %. Alternatively, cocamidopropyl betaine can be sourced from Tinci as TC-CAB 35 having salt content below or equal to about 6.0 wt. %; or from Evonik as TEGO BETAIN F-50 having a sodium chloride content between about 5.80-7.30 wt. %; or from Stepan as AMPHOSOL® HCA-HP having a sodium chloride content about 5.2 wt. %. Alternatively, cocamidopropyl betaine can be sourced from Kensing™ as SensaFoam™ CK PH 12/MB having a sodium chloride content of about 5 wt. %.

Alternatively, or in addition to the zwitterionic surfactant, the personal care composition or the cleansing phase may comprise an amphoteric surfactant.

Alternatively, or in addition to the zwitterionic surfactant, the personal care composition may comprise from about 0.01 wt. % to about 20 wt. % of the amphoteric surfactant; preferably from about 0.1 wt. % to about 10 wt. % of the amphoteric surfactant; more preferably from about 1 wt. % to about 10 wt. % of the amphoteric surfactant; most preferably from about 2 wt. % to about 5 wt. % of the amphoteric surfactant.

Additional amphoteric surfactants suitable for use in the cleansing phase can include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be 3-(dodecyldimethylammonio)-2-hydroxypropane-1-sulfonate or Lauryl hydroxysultaine, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378, each of which is incorporated herein by reference. Amphoacetates and diamphoacetates can also be used.

The amphoteric surfactant included in the personal care composition described herein may be preferably selected from the group consisting of sodium lauroamphoacetate, sodium cocoamphoacetate, disodium lauroamphoacetate, disodium cocodiamphoacetate, and mixtures thereof.

Sodium cocoamphoacetate can be sourced from Stepan as AMPHOSOL® 1C having a sodium chloride content between about 6.5 wt. %. Sodium lauroamphoacetate can be sourced from Colonial Chemical, Inc. as Cola® Teric SLAA having a sodium chloride content between about 6.5 wt. % and about 7.5 wt. %.

Optionally, the cleansing phase may further comprise an additional cosurfactant, for example, a nonionic surfactant, Nonionic surfactants suitable for use in the personal care compositions can include those selected from the group consisting of alkyl ethoxylates, alkyl glucosides, polyglucosides (e.g., alkyl polyglucosides, decyl polyglucosides), polyhydroxy fatty acid amides, alkoxylated fatty acid esters, sucrose esters, amine oxides, or mixtures thereof. Some exemplary nonionic surfactants can include cocamide monoethanolamine, decyl glucoside, or a mixture thereof.

Following the addition of the zwitterionic or amphoteric surfactant, the step of forming the cleansing phase may comprise the following step: optionally adding a cationic deposition polymer.

### Cationic Deposition Polymer

Optionally, the personal care composition may additionally comprise a cationic deposition polymer in the cleansing phase as a deposition aid for the benefit agents described herein.

Suitable cationic deposition polymers for use in the compositions may contain cationic nitrogen-containing moieties such as quaternary ammonium moieties. Non-limiting examples of cationic deposition polymers for use in the personal care composition include cationic cellulose derivatives. Preferred cationic cellulose polymers are the salts of hydroxyethyl cellulose reacted with trimethyl ammonium substituted epoxide, referred to in the industry (CTFA) as Polyquaternium 10 which are available from Amerchol Corp. (Edison, N.J., USA) in their Polymer KG, JR and LR series of polymers with the most preferred being KG-30M. Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

The cationic deposition polymers of the personal care composition may have a cationic charge density from about 0.8 meq/g to about 2.0 meq/g, alternatively from about 1.0 meq/g to about 1.5 meq/g.

The personal care composition or the cleansing phase may comprise from about 0.01% to about 5%, preferably from about 0.1% to about 2%, more preferably from about 0.2% to about 1%, most preferably from about 0.3% to about 1% by weight of the personal care composition, of a cationic deposition polymer.

In an aspect, the personal care composition or the cleansing phase may comprise from about 0.01% to about 5%, preferably from about 0.1% to about 2%, more preferably from about 0.2% to about 1%, most preferably from about 0.3% to about 0.5% by weight of the personal care composition, of guar hydroxypropyltrimonium chloride.

Anionic Surfactant being Substantially Free of Sulfate

After adding the rheology modifier under a high shear or a turbine agitation, the step of forming the cleansing phase comprises the step of adding the anionic surfactant being substantially free of sulfate, preferably the N-acyl amino acid surfactant, most preferably the N-acyl alaninate surfactant.

Suitable anionic surfactants that are substantially free of sulfates can include sodium, ammonium or potassium salts of isethionates; sodium, ammonium or potassium salts of sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of lactates; sodium, ammonium or potassium salts of lactylates; sodium, ammonium or potassium salts of taurates; sodium, ammonium or potassium salts of alaninates; and combinations thereof.

The personal care composition may comprise from about 5 wt. % to about 20 wt. % of the anionic surfactant that is substantially free of sulfates; preferably from about 7 wt. % to about 16 wt. % of the anionic surfactant that is substantially free of sulfates; more preferably from about 9 wt. % to about 13 wt. % of the anionic surfactant that is substantially free of sulfates; most preferably from about 10 wt. % to about 13 wt. % of the anionic surfactant that is substantially free of sulfates.

Non-limiting examples of isethionate surfactants can include sodium lauroyl isethionate, sodium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, sodium cocoyl isethionate, sodium cocoyl methyl isethionate, a blend of stearic acid and sodium cocoyl isethionate, ammonium cocoyl isethionate, ammonium cocoyl methyl isethionate, and mixtures thereof.

Non-limiting examples of sulfonates can include alpha olefin sulfonates, linear alkylbenzene sulfonates, sodium laurylglucosides hydroxypropylsulfonate, and combinations thereof.

Non-limiting examples of sulfosuccinate surfactants can include disodium N-octadecyl sulfosuccinate, disodium lauryl sulfosuccinate, diammonium lauryl sulfosuccinate, sodium lauryl sulfosuccinate, disodium laureth sulfosuccinate, tetrasodium N-(1,2-dicarboxyethyl)-N-octadecyl sulfosuccinate, diamyl ester of sodium sulfosuccinic acid, dihexyl ester of sodium sulfosuccinic acid, dioctyl esters of sodium sulfosuccinic acid, and combinations thereof.

Non-limiting examples of sulfoacetates can include sodium lauryl sulfoacetate, ammonium lauryl sulfoacetate, and combination thereof.

Non-limiting examples of acyl glycinates can include sodium cocoyl glycinate, sodium lauroyl glycinate, and combination thereof.

Non-limiting examples of sarcosinate surfactants can include sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroyl glutamate/lauroyl sarcosinate, lauroyl sarcosinate, isopropyl lauroyl sarcosinate, potassium cocoyl sarcosinate, potassium lauroyl sarcosinate, sodium oleoyl sarcosinate, sodium palmitoyl sarcosinate, TEA-lauroyl sarcosinate, TEA-oleoyl sarcosinate, TEA-palm kernel sarcosinate, and mixtures thereof.

Non-limiting examples of acyl glutamates can be selected from the group consisting of sodium cocoyl glutamate, disodium cocoyl glutamate, ammonium cocoyl glutamate, diammonium cocoyl glutamate, sodium lauroyl glutamate, disodium lauroyl glutamate, sodium cocoyl hydrolyzed wheat protein glutamate, disodium cocoyl hydrolyzed wheat protein glutamate, potassium cocoyl glutamate, dipotassium cocoyl glutamate, potassium lauroyl glutamate, dipotassium lauroyl glutamate, potassium cocoyl hydrolyzed wheat protein glutamate, dipotassium cocoyl hydrolyzed wheat protein glutamate, sodium capryloyl glutamate, disodium capryloyl glutamate, potassium capryloyl glutamate, dipotassium capryloyl glutamate, sodium undecylenoyl glutamate, disodium undecylenoyl glutamate, potassium undecylenoyl glutamate, dipotassium undecylenoyl glutamate, disodium hydrogenated tallow glutamate, sodium stearoyl glutamate, disodium stearoyl glutamate, potassium stearoyl glutamate, dipotassium stearoyl glutamate, sodium myristoyl glutamate, disodium myristoyl glutamate, potassium myristoyl glutamate, dipotassium myristoyl glutamate, sodium cocoyl/hydrogenated tallow glutamate, sodium cocoyl/palmoyl/sunfloweroyl glutamate, sodium hydrogenated tallowoyl glutamate, sodium olivoyl glutamate, disodium olivoyl glutamate, sodium palmoyl glutamate, disodium palmoyl glutamate, TEA-cocoyl glutamate, TEA-hydrogenated tallowoyl glutamate, TEA-lauroyl glutamate, and mixtures thereof.

Non-limiting example of lactates can include sodium lactate.

Non-limiting examples of lactylates can include sodium lauroyl lactylate, sodium cocoyl lactylate, and combination thereof.

Non-limiting examples of acyl taurates can include sodium methyl cocoyl taurate, sodium methyl lauroyl taurate, sodium methyl oleoyl taurate, and combinations thereof.

Non-limiting example of acyl alaninates can include sodium cocoyl alaninate, sodium lauroyl alaninate, sodium N-dodecanoyl-1-alaninate, and combinations thereof.

In that disclosure, alkyl is defined as a saturated or unsaturated, straight or branched alkyl chain with 6 to 30 carbon atoms, preferably with 8 to 22 carbon atoms, more preferably with 9 to 18 carbon atoms. In that case, acyl is defined as of formula R—C(O)—, wherein R is a saturated or unsaturated, straight or branched alkyl or alkenyl, preferably alkyl chain with 6 to 30 carbon atoms, preferably with 8 to 22 carbon atoms, more preferably with 9 to 18 carbon atoms.

Preferably, the anionic surfactant that is substantially free of sulfates is an N-acyl amino acid surfactant. Suitable N-acyl amino acid surfactants may include sodium, ammonium or potassium salts of sarcosinates; sodium, ammonium or potassium salts of glutamates; sodium, ammonium or potassium salts of alaninates; sodium, ammonium or potassium salts of glycinates; and combinations thereof.

Most preferably, the anionic N-acyl amino acid surfactant comprises a N-acyl alaninate surfactant.

The anionic surfactant that is substantially free of sulfates, preferably anionic N-acyl amino acid surfactant may be added relatively slowly over about 2 to about 10 minutes to the main mixing vessel at a side sweep speed from about 5 to about 10 rpm and a turbine speed from about 10 to about 20 rpm.

When the cleansing phase comprises a cationic deposition polymer, the relatively slow addition of the anionic surfactant that is substantially free of sulfates can help to prevent any coacervate formation between the cationic deposition polymer and the anionic surfactant. The coacervate formation may cause instability of the cleansing phase.

### N-Acyl Alaninate Surfactant

The N-acyl alaninate surfactant may be selected from the group consisting of sodium cocoyl alaninate, triethylamine cocoyl alaninate, sodium lauroyl alaninate, sodium N-dodecanoyl-1-alaninate, and combinations thereof.

As a preferred aspect, the N-acyl alaninate surfactant comprises sodium cocoyl alaninate. Sodium cocoyl alaninate is an anionic amino acid from alanine and coconut fatty acid derived surfactant from nature. The N-acyl alaninate surfactant such as sodium cocoyl alaninate is sulfate free. The material is biodegradable, hypoallergenic, mild to skin and eye. Sodium cocoyl alaninate can help for delivering mild cleansing which imparts pleasant moisturizing feel after drying. Sodium cocoyl alaninate can be sourced from Ajinomoto as Amilite™ ACS-12. Alternatively, sodium cocoyl alaninate can be sourced from Sino-Lion as Eversoft™ ACS-30S having a content of sodium chloride between about 4-6 wt. %, or Eversoft™ ACS having a content of sodium chloride of less than about 1 wt. %, around about 0.2 wt. %.

The personal care composition may comprise from about 5 wt. % to about 20 wt. % of the N-acyl alaninate surfactant; preferably from about 7 wt. % to about 16 wt. % of the N-acyl alaninate surfactant; more preferably from about 9 wt. % to about 13 wt. % of the N-acyl alaninate surfactant; most preferably from about 10 wt. % to about 13 wt. % of the N-acyl alaninate surfactant.

The personal care composition may comprise from about 5 wt. % to about 20 wt. % of sodium cocoyl alaninate; preferably from about 7 wt. % to about 16 wt. % of sodium cocoyl alaninate; more preferably from about 9 wt. % to about 13 wt. % of sodium cocoyl alaninate; most preferably from about 10 wt. % to about 13 wt. % of sodium cocoyl alaninate.

The personal care composition or the cleansing phase may not comprise any additional anionic surfactants being not an alaninate surfactant.

Alternatively, the personal care composition or the cleansing phase comprises an N-acyl alaninate surfactant, and the N-acyl alaninate surfactant has a relative carbon-chain length distribution in the N-acyl alaninate surfactant that is such that:
a mixture of C8 and C10 chains within the relative carbon-chain length distribution is between about 17 wt. % to about 25 wt. %, preferably between about 18 wt. % to about 22 wt. %, more preferably between about 18 wt. % to about 20 wt. %;
C12 chain within the relative carbon-chain length distribution is between about 58 wt. % to about 74 wt. %, preferably between about 62 wt. % to about 71 wt. %, more preferably between about 67 wt. % to about 70 wt. %;
C14 chain within the relative carbon-chain length distribution is between about 8 wt. % to about 13 wt. %, preferably between about 9 wt. % to about 12 wt. %, more preferably between about 10 wt. % to about 12 wt. %; and
C16 chain within the relative carbon-chain length distribution is between about 1 wt. % to about 4 wt. %, preferably between about 2 wt. % to about 4 wt. %, more preferably between about 2 wt. % to about 3 wt. %.

The relative concentrations for each carbon-chain length mentioned herein are by weight of the N-acyl alaninate surfactant and can be measured according to the Carbon-chain length distribution Test Method for an N-acyl alaninate surfactant as disclosed herein.

Gas chromatography of nonvolatile species such as N-acyl alaninate surfactant requires derivatization such as trimethylsilylation with O-Bis(trimethylsilyl)trifluoroacetamide with 1% Trimethylchlorosilane (BSTFA-TMCS). The addition of a trimethylsilyl group to the carboxylic acid function of the N-acyl alaninate surfactant can help in volatility and improve peak shape. Lauroyl alanine standard has been used as a reference for peak identification.

In other words, the relative carbon-chain length distribution in the N-acyl alaninate surfactant is such that: a mixture of C8 and C10 chains within the relative carbon-chain length distribution is between about 17 wt. % to about 25 wt. %; C12 chain within the relative carbon-chain length distribution is between about 58 wt. % to about 74 wt. %; C14 chain within the relative carbon-chain length distribution is between about 8 wt. % to about 13 wt. %; and C16 chain within the relative carbon-chain length distribution is between about 1 wt. % to about 4 wt. %.

Higher levels of C8 and C10 chains and lower levels of C14 could prevent unstable personal care compositions, especially unstable cleansing phases.

Preferably, the relative carbon-chain length distribution in the N-acyl alaninate surfactant may be such that: a mixture of C8 and C10 chains within the relative carbon-chain length distribution is between about 18 wt. % to about 22 wt. %; C12 chain within the relative carbon-chain length distribution is between about 62 wt. % to about 71 wt. %; C14 chain within the relative carbon-chain length distribution is between about 9 wt. % to about 12 wt. %; and C16 chain within the relative carbon-chain length distribution is between about 2 wt. % to about 4 wt. %.

More preferably, the relative carbon-chain length distribution in the N-acyl alaninate surfactant may be such that: a mixture of C8 and C10 chains within the relative carbon-chain length distribution is between about 18 wt. % to about 20 wt. %; C12 chain within the relative carbon-chain length distribution is between about 67 wt. % to about 70 wt. %; C14 chain within the relative carbon-chain length distribution is between about 10 wt. % to about 12 wt. %; and C16 chain within the relative carbon-chain length distribution is between about 2 wt. % to about 3 wt. %.

Alternatively, the relative carbon-chain length distribution in the N-acyl alaninate surfactant may include a minimum amount of the mixture of C8 and C10 chains within the relative carbon-chain length distribution of at least about 17 wt. %, preferably of at least about 18 wt. % and a weight ratio of (C14+C16)/(C8+C10)<1.0.

In other words, the relative carbon-chain length distribution in the N-acyl alaninate surfactant may include a mixture of C8 and C10 chains within the relative carbon-chain length distribution is between about 17 wt. % to about 25 wt. %, preferably between about 18 wt. % to about 22 wt. %, more preferably between about 18 wt. % to about 20 wt. %; and a weight ratio of (C14+C16)/(C8+C10)<1.0, preferably a weight ratio of (C14+C16)/(C8+C10)<0.9, more preferably a weight ratio of (C14+C16)/(C8+C10)<0.7.

The relative carbon-chain length distribution in the N-acyl alaninate surfactant may be substantially free of C18 and C18:1 chains. Namely, the relative carbon-chain length distribution in the N-acyl alaninate surfactant may comprise less than about 0.1 wt. % or less than 0.05 wt. % or less than 0.02 wt. % or less than 0.01 wt. % or free of C18 and C18:1 chains. C18:1 is a C18 chain with one double bond.

Also, the relative carbon-chain length distribution in the N-acyl alaninate surfactant may be substantially free of C6 chains. Namely, the relative carbon-chain length distribution in the N-acyl alaninate surfactant may comprise less than about 0.1 wt. % or less than 0.05 wt. % or less than 0.02 wt. % or less than 0.01 wt. % or free of C6 chains.

The N-acyl alaninate surfactant may be a sodium, potassium, ammonium, or triethylamine N-acyl alaninate.

By utilizing a sulfate-free surfactant system, namely the N-acyl alaninate surfactant with the optimal relative carbon-chain length distribution as recited herein and specific structuring system, the composition achieves the desired balance between structure and lamellar vesicle formation, lather and resulting in effective skin cleansing and deposition of benefit agents.

In that respect, the personal care composition may include one or more lamellar vesicles, or multiple lamellar vesicles.

The N-acyl alaninate surfactant may be selected from the group consisting of sodium cocoyl alaninate, triethylamine cocoyl alaninate, and combinations thereof.

As a preferred aspect, the N-acyl alaninate surfactant comprises sodium cocoyl alaninate with the specific relative carbon-chain length distribution in the N-acyl alaninate surfactant as recited herein.

N-acyl alaninate surfactants are typically prepared by the reaction of L-alanine and a respective mixture of aliphatic fatty acid chlorides with the recited relative carbon-chain length distribution. For this, the blend of aliphatic fatty acid chlorides with the recited relative carbon-chain length distribution may be obtained from the respective mixture of aliphatic fatty acids according to known process as described in Bauer, S. T.; JAOCS Vol 23, Issue 1, January 1946, pages 1-5, which is incorporated by reference.

Alternatively, N-acyl alaninate surfactants can be made with a Dean-Stark method by combining L-alanine with a blend of fatty acid methyl esters with the corresponding relative carbon-chain length distribution. In that case, methanol is continuously removed from the reaction mixture. Preparation of N-acyl alaninate surfactants with different carbon-chain length distributions is described, see for instance US 2022/0401328 A1, which is incorporated herein by reference.

The personal care composition may comprise from about 5 wt. % to about 20 wt. % of the N-acyl alaninate surfactant; preferably from about 7 wt. % to about 16 wt. % of the N-acyl alaninate surfactant; more preferably from about 9 wt. % to about 13 wt. % of the N-acyl alaninate surfactant; most preferably from about 10 wt. % to about 13 wt. % of the N-acyl alaninate surfactant.

The concentrations mentioned here are total concentration ranges in case more than one N-acyl alaninate surfactant is present. The specified ranges are provided by weight and relate to the total weight of the personal care composition. The concentrations mentioned hereinbefore apply to any carbon-chain length distribution in the N-acyl alaninate surfactant defined herein.

The personal care composition may comprise from about 5 wt. % to about 20 wt. % of sodium cocoyl alaninate; preferably from about 7 wt. % to about 16 wt. % of sodium cocoyl alaninate; more preferably from about 9 wt. % to about 13 wt. % of sodium cocoyl alaninate; most preferably from about 10 wt. % to about 13 wt. % of sodium cocoyl alaninate.

The personal care composition or the cleansing phase may not comprise any additional anionic surfactants being not a N-acyl alaninate surfactant.

The personal care composition or the cleansing phase may comprise an additional N-acyl alaninate surfactant, wherein the additional N-acyl alaninate surfactant is selected from the group consisting of sodium lauroyl alaninate, sodium N-dodecanoyl-1-alaninate, and combinations thereof.

### Emulsifying Agent

After adding the rheology modifier, the zwitterionic or amphoteric surfactant, optionally the cationic deposition polymer, and the anionic surfactant being substantially free of sulfate, the step of forming the cleansing phase may comprise the following step: adding the emulsifying agent directly or from a first jacketed vessel to the main mixing vessel.

Alternatively, after adding the zwitterionic or amphoteric surfactant, and optionally the cationic deposition polymer and the preservative, the step of forming the cleansing phase may comprise the following steps, preferably in that order:
adding the emulsifying agent directly or from a first jacketed vessel to the main mixing vessel;
adding the rheology modifier under a high shear or a turbine agitation.

The emulsifying agent may be a glyceryl ester and/or a non-ionic emulsifier having an HLB of from about 3.4 to about 13.0. The emulsifying agent will be described more in detailed below.

The emulsifying agent may be added directly to the main mixing vessel. This is typically the case when the emulsifying agent is liquid at 25° C. and ambient conditions, e.g., trideceth-3.

Alternatively or in addition to, the emulsifying agent may be added from a first jacketed vessel to the main mixing vessel, as shown in a FIG. 1.

Prior to be added or dispersed in the main mixing vessel, the emulsifying agent may be melted in the first jacketed vessel.

In that aspect, the emulsifying agent may be melted in the first jacketed vessel at a temperature above a melting point of the emulsifying agent. Indeed, the emulsifying agent may be typically melted when the emulsifying agent is solid at 25° C. and ambient conditions, or liquid to semi-solid at 25° C. and ambient conditions.

The emulsifying agent may be a glyceryl ester. The glyceryl ester may be preferably selected from glyceryl laurate, glyceryl caprate, glyceryl caprylate, glyceryl caprylate/caprate, glyceryl stearate, and a mixture thereof. As a preferred aspect, the emulsifying agent may comprise glyceryl caprylate/caprate.

The emulsifying agent may comprise a glyceryl ester, wherein the glyceryl ester is selected from glyceryl laurate, glyceryl caprate, glyceryl caprylate, glyceryl caprylate/caprate, glyceryl stearate, and a mixture thereof, wherein the glyceryl ester is melted in the first jacketed vessel at a temperature above a melting point of the glyceryl ester, more preferably from about 30° C. to about 70° C., or from about 38° C. to about 65° C., most preferably from about 40° C. to about 63° C.

Preferably, the emulsifying agent may comprise glyceryl caprylate/caprate, wherein glyceryl caprylate/caprate is melted in the first jacketed vessel at a temperature above a melting point of glyceryl caprylate/caprate, more preferably from about 35° C. to about 50° C., or from about 38° C. to about 45° C., most preferably from about 38° C. to about 42° C.

Alternatively, the emulsifying agent may comprise a glyceryl ester, wherein the glyceryl ester is selected from glyceryl laurate, glyceryl caprate, glyceryl caprylate, glyceryl caprylate/caprate, glyceryl stearate, and a mixture thereof, wherein the glyceryl ester is melted in the first jacketed scrape wall vessel at a temperature above a melting point of the glyceryl ester, more preferably from about 30° C. to about 70° C., or from about 38° C. to about 65° C., most preferably from about 40° C. to about 63° C.

Preferably, the emulsifying agent may comprise glyceryl caprylate/caprate, wherein glyceryl caprylate/caprate is melted in the first jacketed scrape wall vessel at a temperature above a melting point of glyceryl caprylate/caprate, more preferably from about 35° C. to about 50° C., or from about 38° C. to about 45° C., most preferably from about 38° C. to about 42° C.

Glyceryl caprylate/caprate can be sourced from Stepan as Stepan-Mild® GCC. In that case, the melting point of glyceryl caprylate/caprate is about 24-30° C. and the appearance of glyceryl caprylate/caprate is pale yellow liquid to white semi-solid.

In an alternative aspect, as set out above, the rheology modifier may be in a powder form, e.g. xanthan gum. As seen above, such rheology modifier may be added using a high shear homogenizer such as Quadro® HV high shear mixer homogenizer.

Alternatively, the rheology modifier may be added in the first jacketed vessel together with the emulsifying agent. In the first jacketed vessel, the rheology modifier and the emulsifying agent may form a slurry to be added together to the main mixing vessel. Such slurry can help preventing agglomeration of the rheology modifier like xanthan gum in the cleansing phase.

The personal care composition may comprise from about 0.3 wt. % to about 1.5 wt. % of hydroxypropyl starch phosphate and from about 0.1 wt. % to about 0.5 wt. % of xanthan gum, preferably from about 0.3 wt. % to about 1.0 wt. % of hydroxypropyl starch phosphate and from about 0.1 wt. % to about 0.4 wt. % of xanthan gum, more preferably from about 0.75 wt. % to about 0.90 wt. % of hydroxypropyl starch phosphate and from about 0.2 wt. % to about 0.3 wt. % of xanthan gum.

In that aspect, hydroxypropyl starch phosphate may be added to the main mixing vessel under a high shear or a turbine agitation. Xanthan gum may be added as a slurry with the emulsifying agent in the first jacketed vessel, preferably wherein the emulsifying agent comprises glyceryl caprylate/caprate.

In that aspect, the emulsifying agent may comprise glyceryl caprylate/caprate, wherein glyceryl caprylate/caprate is melted in the first jacketed scrape wall vessel at a temperature above a melting point of glyceryl caprylate/caprate, more preferably from about 35° C. to about 50° C., or from about 38° C. to about 45° C., most preferably from about 38° C. to about 42° C.

### Jacketed Vessel

A jacketed vessel is s a type of container or tank used in various industrial processes. It is designed with an outer jacket surrounding the main vessel, creating a space between the two layers. This space is typically filled with a heat transfer fluid, such as steam or hot water, which circulates through the jacket to provide heating or cooling to the contents of the vessel or tank.

The purpose of the jacket is to control and regulate the temperature of the material inside the vessel or tank. By circulating the temperature-regulating fluid through the jacket, heat can be transferred to or from the material inside the vessel, allowing for precise temperature control during processing.

Preferably, the first jacketed vessel may be a jacketed scrape wall vessel or a jacketed vessel including a propeller turbine equipped with a heating or cooling jacket.

A jacketed scrape wall vessel is a type of vessel used for processing and storing fluids in various industries. It combines the features of a jacketed vessel and a scrape surface mixer or agitator. The vessel is equipped with a jacket for heating or cooling the contents, and it has an internal scraping mechanism to enhance mixing, heat transfer, and prevent product buildup on the vessel walls.

A jacketed scrape wall vessel typically may consist of a cylindrical or rectangular vessel made of stainless steel or other suitable materials. The jacketed scrape wall vessel is equipped with a heating or cooling jacket, which is an outer shell surrounding the main vessel. The jacket has inlet and outlet connections for the circulation of a temperature control medium, such as steam, hot water, or chilled water.

Inside the jacketed scrape wall vessel, there may be a scraping mechanism or agitator that consists of rotating blades or paddles. The blades are attached to a central shaft that extends into the vessel. The scraping mechanism is driven by a motor located outside the vessel.

When the jacketed scrape wall vessel is in operation, the scraping mechanism rotates, causing the blades to scrape along the inner walls of the vessel. This scraping action promotes mixing and prevents product buildup on the vessel walls. It can also enhance heat transfer between the vessel contents and the jacketed surface.

The scraped surface design helps ensure a homogeneous product and efficient transfer of the contents.

Alternatively, the first jacketed vessel may be a jacketed vessel including a propeller turbine equipped with a heating or cooling jacket.

The first jacketed vessel may typically consist of three main components: the inner vessel, the jacket, and the agitator system. The inner vessel is the primary container that holds the product or material to be processed. It can be made of various materials such as stainless steel, glass, or other corrosion-resistant materials, depending on the specific application.

The jacket, which surrounds the inner vessel, is a hollow space or cavity through which a heating or cooling medium flows. The jacket is usually made of stainless steel and is designed to withstand the pressure and temperature requirements of the process. It is connected to inlet and outlet ports, allowing the circulation of the temperature control medium.

The agitator system in the jacketed vessel typically may include a propeller turbine or impeller, which is mounted on a rotating shaft. The propeller turbine is responsible for mixing and agitating the contents inside the vessel, ensuring uniform temperature distribution and efficient heat transfer. The agitator system can be driven by an electric motor or another power source.

In any cases, when heating is required, a temperature control medium such as steam or hot water may be circulated through the jacket, transferring heat to the jacket vessel contents. If cooling is needed, chilled water or a refrigerant is circulated through the jacket, extracting heat from the vessel contents. The jacketed design allows for efficient and controlled heating or cooling of the product.

Once the desired melting of the emulsifying agent is complete, the first jacketed vessel contents can be discharged through a transfer pump located at the bottom of the vessel and an outlet valve located downstream the conducting line to the main mixing vessel.

The personal care composition may comprise from about 1 wt. % to about 3 wt. % of the emulsifying agent; preferably from about 1 wt. % to about 2.75 wt. % of the emulsifying agent; more preferably from about 1 wt. % to about 2.5 wt. % of the emulsifying agent.

The emulsifying agent is a glyceryl ester. Alternatively, or in addition, the emulsifying agent is a non-ionic emulsifier having an HLB of from about 3.4 to about 13.0.

The personal care composition may comprise from about 1 wt. % to about 3 wt. % of the glyceryl ester; preferably from about 1 wt. % to about 2.75 wt. % of the glyceryl ester; more preferably from about 1 wt. % to about 2.5 wt. % of the glyceryl ester.

The glyceryl ester may be preferably selected from glyceryl laurate, glyceryl caprate, glyceryl caprylate, glyceryl caprylate/caprate, glyceryl stearate, and a mixture thereof.

Other suitable glyceryl esters may be selected such as the glyceryl esters containing C8-C10 mono- di- and tri-glycerides which are different from C8-C10 mono-dicaprylate 1,2,3-propanetriol.

As a preferred aspect, the emulsifying agent comprises glyceryl caprylate/caprate. Glyceryl caprylate/caprate is mild and substantially free of polyethyleneglycol (PEG), Ethylene Oxide/Propylene Oxide (EO/PO), and Nitrogen.

The multifunctional benefits can include yield generation for suspension of particles at relatively high temperature for product stability, viscosity modifier, scalp skin moisturization, wet and dry conditioning, and potential enhanced depo of soluble active. Glyceryl caprylate/caprate can be sourced from Stepan as Stepan-Mild® GCC.

The personal care composition may comprise from about 0.5 wt. % to about 5 wt. % of glyceryl caprylate/caprate; preferably from about 1 wt. % to about 2.75 wt. % of glyceryl caprylate/caprate; more preferably from about 1 wt. % to about 2.5 wt. % glyceryl caprylate/caprate.

Alternatively, or in addition to a glyceryl ester, preferably glyceryl caprylate/caprate, the emulsifying agent is a non-ionic emulsifier having an HLB of from about 3.4 to about 13.0, preferably about 3.4 to about 8.0.

The personal care composition may comprise from about 1 wt. % to about 3 wt. % of the non-ionic emulsifier having an HLB of from about 3.4 to about 13.0; preferably from about 1 wt. % to about 2.75 wt. % of the non-ionic emulsifier having an HLB of from about 3.4 to about 13.0; more preferably from about 1 wt. % to about 2.5 wt. % of the non-ionic emulsifier having an HLB of from about 3.4 to about 13.0.

The balance between the hydrophilic and lipophilic moieties in a surfactant molecule is used as a method of classification (hydrophile-lipophile balance, HLB). The HLB values for commonly-used surfactants are readily available in the literature (e.g., HLB Index in McCutcheon's Emulsifiers and Detergents, MC Publishing Co., 2004). Another way of obtaining HLB values is to estimate by calculations. The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20. Other examples of how to calculate HLB values are described by Davies in Interfacial Phenomena, 2nd Edition, Academic Press, London, 1963 and by Lin in J. Phys. Chem. 76, 2019-2013, 1972.

The non-ionic emulsifier having an HLB of from about 3.4 to about 13.0 may preferably comprise trideceth-3 or trideceth-4.

As a preferred aspect, the emulsifying agent may comprise trideceth-3. In that case, the personal care composition may comprise from about 0.5 wt. % to about 5 wt. % of trideceth-3; preferably from about 1 wt. % to about 2.75 wt. % of trideceth-3; more preferably from about 1 wt. % to about 2.5 wt. % trideceth-3.

The non-ionic emulsifier can help to increase the Carreau zero shear viscosity and thus improve the structure and stability of the personal care composition at a specified pH range described more in detailed below.

In any cases, the personal care composition may comprise a weight ratio of the N-acyl alaninate surfactant to the emulsifying agent that is greater than about 5:1 to about 15:1; preferably from about 5.5:1 to about 14:1; more preferably from about 5.6:1 to about 7:1.

With the levels of the emulsifying agent or a weight ratio of the N-acyl alaninate surfactant to the emulsifying agent as set out hereinbefore, acceptable lather can be obtained.

The personal care composition may comprise a weight ratio of the N-acyl alaninate surfactant to the glyceryl ester that is greater than about 5:1 to about 15:1; preferably from about 5.5:1 to about 14:1; more preferably from about 5.6:1 to about 7:1.

As a most preferred aspect, the personal care composition may comprise a weight ratio of sodium cocoyl alaninate to glyceryl caprylate/caprate that is greater than about 5:1 to about 15:1; preferably from about 5.5:1 to about 14:1; more preferably from about 5.6:1 to about 7:1.

Glyceryl caprylate/caprate at the levels set out hereinabove or when combined with sodium cocoyl alaninate at a recited weight ratio can help to improve the lather properties of the composition.

Alternatively, the personal care composition may comprise a weight ratio of the N-acyl alaninate surfactant to the non-ionic emulsifier having an HLB of from about 3.4 to about 13.0 that is greater than about 5:1 to about 15:1; preferably from about 5.5:1 to about 14:1; more preferably from about 5.6:1 to about 7:1.

The personal care composition may comprise a weight ratio of sodium cocoyl alaninate to trideceth-3 that is greater than about 5:1 to about 15:1; preferably from about 5.5:1 to about 14:1; more preferably from about 5.6:1 to about 7:1.

### Preservative

The personal care composition may comprise from about 0.01% to about 1.0%, preferably from about 0.02% to about 0.4%, more preferably from about 0.05% to about 0.2%, most preferably from about 0.05% to about 0.1% of a preservative by weight of the composition.

The preservative may include a salicylate salt and a benzoate salt, wherein a total amount of the salicylate salt and the benzoate salt is from about 0.2% to about 1.0%, preferably from about 0.5% to about 0.90%, more preferably from about 0.75% to about 0.85%, by weight of the composition.

The weight ratio of the salicylate salt to the benzoate salt may be from about 1:1.10 to about 1:1.20, preferably from about 1:1.125 to about 1:1.175.

The salicylate salt may be sodium salicylate. The benzoate salt may be sodium benzoate.

Following the addition of the emulsifying agent, the step of forming the cleansing phase may comprise the following step: optionally adding a preservative, wherein the preservative includes a salicylate salt and a benzoate salt, wherein a total amount of the salicylate salt and the benzoate salt is from about 0.2% to about 1.0%, preferably from about 0.5% to about 0.90%, more preferably from about 0.75% to about 0.85%, by weight of the personal care composition.

Alternatively, following the addition of the zwitterionic or amphoteric surfactant, the step of forming the cleansing phase may comprise the following steps, preferably in that order:
optionally adding a cationic deposition polymer, wherein the cleansing phase comprises from about 0.01% to about 5%, preferably from about 0.1% to about 2%, more preferably from about 0.2% to about 1%, most preferably from about 0.3% to about 1% by weight of the personal care composition, of guar hydroxylpropyltrimonium chloride; and
optionally adding a preservative, wherein the preservative includes a salicylate salt and a benzoate salt, wherein a total amount of the salicylate salt and the benzoate salt is from about 0.2% to about 1.0%, preferably from about 0.5% to about 0.90%, more preferably from about 0.75% to about 0.85%, by weight of the personal care composition.
   pH

The step of forming the cleansing phase may comprise the additional following steps after adding the emulsifying agent, and optionally the preservative, preferably in that order:
adjusting the pH, preferably with citric acid; and
optionally adding a perfume.

Alternatively, the step of forming the cleansing phase may comprise the additional following steps after adding the anionic surfactant that is substantially free of sulfates, or the anionic N-acyl amino acid surfactant, preferably in that order:
adjusting the pH, preferably with citric acid; and
optionally adding a perfume.

The pH of the personal care composition is from about 4.0 to about 5.5, preferably from about 4.2 to about 5.3, more preferably from about 4.5 to about 5.2, most preferably from about 4.8 to about 5.2. The pH of the personal care composition can help to provide a structured cleansing phase.

A variety of compounds may be used to adjust the pH value of a composition. Such suitable compounds can include, but are not limited to, citric acid, acetic acid, hydrochloric acid, triethylamine, diethylamine, ethylamine, monoethanol amine, diethanol amine, triethanol amine and any mixtures thereof. The personal care composition may comprise greater than about 0% to about 3% of the pH adjusting agent by weight of the composition, preferably wherein the pH adjusting agent comprises citric acid.

Set up the pH of the personal care composition as recited herein can help to prevent phase separation of the personal care composition. Then, the surfactant levels and/or can be optimized as described herein for building and improving the rheology or viscosity profile of the personal care composition.

### Perfume

The cleansing phase may include a perfume. The perfume may be added in the main mixing vessel on the batch production line. Indeed, the perfume can help impact the surfactant packing structure and help with the lamellar phase stability of the cleansing phase.

Hence, the perfume may be added in the main mixing vessel on the batch production line quite the opposite of the typical late-variant addition processes where such processes are intended to protect temperature and shear sensitive additives such as perfume, fragrances and organic colorants. In these former processes, temperature and shear sensitive additives such as perfume, fragrances and organic colorants are not added in the main mixing vessel on a batch production line, however on a flow line downstream of the batch production line at room temperature to preserve their fragility.

The personal care composition may further comprise from about 0.01% to about 2% of a perfume by weight of the composition, preferably from about 0.1% to about 1.75% of a perfume by weight of the composition, more preferably from about 0.5% to about 1.6% of a perfume by weight of the composition, even more preferably from about 0.8% to about 1.5% of a perfume by weight of the composition.

Typically, the perfume may be a blend of perfumes and aroma chemicals. As used herein, “fragrance” is used to indicate any odoriferous material.

A wide variety of chemicals are known as fragrances in the perfume, including alcohols, aldehydes, ketones, and esters. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release. The fragrances herein may be relatively simple in their chemical make-up, comprising a single chemical, or may comprise highly sophisticated complex mixtures of natural and synthetic chemical components, all chosen to provide any desired odor.

Suitable fragrances are also disclosed in U.S. Pat. Nos. 4,145,184, 4,209,417, 4,515,705, and 4,152,272, each of which is incorporated herein by reference. Non-limiting examples of fragrances include animal fragrances such as musk oil, civet, castoreurn, ambergris, plant fragrances such as nutmeg extract, cardomon extract, ginger extract, cinnamon extract, patchouli oil, geranium oil, orange oil, mandarin oil, orange Hower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract.

Other examples of suitable fragrances include, but are not limited to, chemical substances such as acetophenone, adoxal, aldehyde C-12, aldehyde C-14, aldehyde C-18, allyl caprylate, ambroxan, amyl acetate, dimethylindane derivatives, «-amylcinnamic aldehyde, anethole, anisaldehyde, benzaldehyde, borneol, butyl acetate, camphor, carbitol, cinnamaldehyde, cinnamyl acetate, cinnamyl alcohol, cis-3-hexanol and ester derivatives, cis-3-bexenyl methyl carbonate, citral, citronnellol and ester derivatives, cumin aldehyde, cyclamen aldehyde, cyclogalbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltoiide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dibydrojasmonate, methyl eugenol, methyl ionone, methyl-α-naphthyl ketone, methylphenylcarbinyl acetate, mugetanol, γ-nonalactone, octanal, phenyl ethyl acetate, phenylacetaldehyde dimethyl acetate, phenoxyethyl isobutyrate, phenyl ethyl alcohol, pinenes, sandalore, santaiol, stemone, thymol, terpenes, triplal, triethyl citrate, 3,3,5-trimethylcyclohexanol, γ-undecalactone, undecenal, vanillin, veloutone, verdox, and mixtures thereof.

### Lamellar Structure

The cleansing phase of the personal care composition is an aqueous structured surfactant phase. The cleansing phase may be comprised of a structured domain that comprises the surfactants as set out hereinabove. The structured domain may be preferably an opaque structured domain, which is preferably a lamellar phase. The lamellar phase produces lamellar vesicles. The lamellar phase can provide resistance to shear, adequate yield to suspend particles and droplets and at the same time provides long term stability, since it is thermodynamically stable.

The personal care composition may be a structured lamellar composition. The personal care composition may comprise at least a 40% lamellar structure, preferably at least a 50% lamellar structure, more preferably at least a 70% lamellar structure.

Alternatively, the personal care composition may comprise a lamellar phase volume from about 40% to about 100%, preferably from about 50% to about 100%, more preferably from about 70% to about 100% of a lamellar phase volume according to the Ultracentrifugation Method disclosed herein.

As an aspect of the disclosure, the personal care composition disclosed herein may also be substantially free of one or more inorganic electrolytes.

Such one or more inorganic electrolytes include halides of alkaline metals, alkaline earth metals, ammonium and other metals, such as aluminum and zinc; sulphates and phosphates of alkaline metals, alkaline earth metals, ammonium and other metals such has aluminum and zinc; and alkaline metal silicates, among others.

As a preferred aspect, the personal care composition may substantially free of one or more inorganic electrolytes including sodium chloride, potassium chloride, sodium sulphate, potassium sulphate, magnesium chloride, magnesium sulphate, magnesium chloride, magnesium sulphate, zinc sulphate, ammonium chloride, and combinations thereof.

As a more preferred aspect, the personal care composition may substantially free of sodium chloride.

The term “substantially free of one or more inorganic electrolytes” as used herein means less than about 1.25%, less than about 1.2%, less than about 1%, less than about 0.8%, less than about 0.5%, less than about 0.3%, less than about 0.1%, less than about 0.01% or less than an immaterial amount of inorganic electrolytes by weight of the composition.

The term “substantially free of sodium chloride” as used herein means less than about 1.25%, less than about 1.2%, less than about 1%, less than about 0.8%, less than about 0.5%, less than about 0.3%, less than about 0.1%, less than about 0.01% or less than an immaterial amount of sodium chloride by weight of the composition.

When having a low level of one or more inorganic electrolytes; or sodium chloride, the % of lamellar structure could be enhanced. Such improvement could help for providing resistance to shear, adequate yield to suspend particles and droplets and at the same time provides long term stability.

To provide a personal care composition being substantially free of one or more inorganic electrolytes; or sodium chloride, the personal care composition may not comprise any further inorganic electrolyte or sodium chloride added.

The zwitterionic or amphoteric surfactant may have a relatively low content of inorganic electrolytes or sodium chloride. For instance, cocamidopropyl betaine can be sourced from BASF as Dehyton® PK 45 having the sodium chloride content removed, resulting in about 33.05 wt. % dry residue; and about 0.21 wt. % sodium chloride.

### Admixing the Benefit Phase to the Cleansing Phase

The process for manufacturing the personal care composition comprises the step of admixing the benefit phase to the cleansing phase on a flow line downstream of the batch production line. The benefit phase comprises from about 0.1 wt. % to about 50 wt. % of a benefit agent. Then, the resultant personal care composition is recovered.

The flow line is the section of the production line where the product of the main mixing vessel (i.e., the cleansing phase) is pumped out through a pipeline or a conduit, and additional ingredients (e.g., the benefit phase) are continuously added and mixed while the product flows. The phases are mixed as it continuously makes its way through a series of tanks, tubes, or other vessels.

The resulting personal care composition may be pumped out in a storage vessel before being transported into a package filler.

Alternatively, the resulting personal care composition may be transported directly into a package filler, as shown in a FIG. 1 for instance.

The flow line may be seen as a continuous process section that refers to the flow of a single unit of product (the cleansing phase) between every step of the process (admixing the benefit phase, emulsification . . . ) without any break in time, substance, or extend.

The flow line is a continuous process section that is sophisticated and geared to larger-scale operations. Typically, the flow line is well suited for products having complex rheologies and imparting a controlled mixing environment from gentle to intense. Depending on the product state and requirements at each point along the process, a number of unit operations may be employed. For mixing, these unit operations may range from simple inline static mixers to any of a variety of high-shear mixers. Heat transfer can be accomplished by a variety of heat exchanger designs including shell and tube and plate and frame-type designs. The flow line has the advantages to be optimized and produce large volumes.

The resulting mixture may be continuously transferred to a reservoir or the packaging filling line without interruption. This type of production is often used when a continuous and uninterrupted supply of the product is desired, or when the process is more efficient when operated continuously.

The resultant personal care composition may be filled, for example, into a number of single-quantity containers, e.g. consumer disposable or recyclable containers or bottles.

The benefit phase may be provided from a second jacketed vessel and the benefit agent is melted prior to admix the benefit phase to the cleansing phase on the flow line downstream of the batch production line.

Preferably, the benefit phase may be provided from a second jacketed vessel and the benefit agent is melted prior to admix the benefit phase to the cleansing phase on the flow line downstream of the batch production line at a temperature above the melting point of the benefit agent.

More preferably, the benefit phase may be provided from a second jacketed vessel and the benefit agent is melted prior to admix the benefit phase to the cleansing phase on the flow line downstream at a temperature of at least about 35° C. or above about 35° C., or from about 35° C. to about 65° C., or from about 38° C. to about 45° C.

The second jacketed vessel may be a second jacketed scrape well vessel. The vessel is entirely jacketed to ensure temperature control of the benefit agent. Such jacketed vessel or jacketed scrape wall vessel has been described in detail hereinbefore.

Alternatively, the benefit phase may be provided from a scrape wall heat exchanger vessel and the benefit agent is melted before admixing the benefit phase to the cleansing phase on the flow line downstream of the batch production line.

Thus, in this alternative, the benefit phase may be provided from a scrape wall heat exchanger vessel and the benefit agent is melted before admixing the benefit phase to the cleansing phase on the flow line downstream of the batch production line.

Preferably, the benefit phase may be provided from a scrape wall heat exchanger vessel and the benefit agent is melted before admixing the benefit phase to the cleansing phase on the flow line downstream of the batch production line at a temperature above the melting point of the benefit agent.

More preferably, the benefit phase may be provided from a scrape wall heat exchanger vessel and the benefit agent is melted before admixing the benefit phase to the cleansing phase on the flow line downstream of the batch production line at a temperature of at least about 35° C. or above about 35° C., or from about 35° C. to about 65° C., or from about 38° C. to about 45° C.

A non-limiting example of a scrape wall heat exchanger may be a Votator®. The Votator® is a type of scraped surface heat exchanger used in various industries for heating, cooling, crystallization, and other thermal processing applications. It is known for its efficient heat transfer capabilities and its ability to handle viscous or non-Newtonian fluids.

The basic principle of the Votator® is to transfer heat between two fluids while maintaining a scraping action on the heat transfer surface. The heat transfer surface typically consists of a jacketed cylinder or tube, and within the cylinder, there is a rotating shaft with attached scraper blades. The scraped surface is in direct contact with the fluid undergoing heat transfer.

The scrape wall heat exchanger such as the Votator® may be used to cool down the benefit agent to a temperature above a melting point of the benefit agent, preferably at a temperature of at least about 35° C. or above about 35° C., or from about 35° C. to about 65° C., or from about 38° C. to about 45° C.

The benefit phase may be admixed to the cleansing phase at a temperature above a melting point of the benefit agent, preferably at a temperature of at least about 35° C. or above about 35° C. or from about 35° C. to about 65° C., or from about 38° C. to about 45° C.

The benefit agent is defined hereinafter in detail. The process features related to the admixing step of the benefit phase to the cleansing phase apply to any of the benefits agents.

The benefit phase may be admixed to the cleansing phase at a temperature above a melting point of the benefit agent, preferably at a temperature of at least about 35° C. or above about 35° C. or from about 35° C. to about 65° C., or from about 38° C. to about 45° C., most preferably wherein the benefit agent includes shea butter.

The line of the benefit phase on the flow line may be heat traced to ensure that the temperature of the benefit phase remains above the melting point of the benefit agent.

As set out hereinbefore, the main mixing vessel may be preferably jacketed for ensuring a temperature control of the cleansing phase. The temperature control of the cleansing phase can help to control the step of admixing the benefit phase to the cleansing phase at a temperature above a melting point of the benefit agent.

Indeed, in the flow line downstream the batch production line, when admixing the benefit phase to the cleansing phase, a cooler cleansing phase than the benefit phase may impact the temperature of the benefit phase at the admixing point, which might partially solidify the benefit phase before the mixture contacts the mixing device for emulsification.

To prevent such temperature control of the cleansing phase, the mixing device may be positioned just after the admixing of the benefit phase to the cleansing phase.

In case the benefit agent is liquid at at about 25° C. and at ambient conditions, the process for manufacturing the personal care composition may comprise the step of admixing the benefit phase to the cleansing phase on a flow line downstream of the batch production line at about 25° C. and at ambient conditions. The benefit phase may be provided from a second jacketed vessel and the benefit phase is admixed to the cleansing phase on the flow line downstream of the batch production line at at about 25° C. and at ambient conditions.

A series of pumps may transfer the benefit phase into the cleansing phase stream flowing with a conduit, as shown in a FIG. 1 for instance, to the next step: the emulsification. A series of back flow valves may be installed to maintain the stream integrity.

After the step of admixing the benefit phase to the cleansing phase, the benefit phase may be emulsified with the cleansing phase into a mixing device at a temperature above the melting point of the benefit agent, wherein the mixing device is a static mixer unit with a diameter from about 25.4 mm (1 inches) to about 63.5 mm (2.5 inches), preferably from about 38.1 mm (1.5 inches) to about 57.1 (2.25 inches), more preferably from about 44.4 mm (1.75 inches) to about 50.8 mm (2 inches) and including from about 8 to about 20 mixing elements, preferably from about 9 to about 15 mixing elements, more from about 10 to 13 mixing elements.

Most preferably, the benefit phase may be emulsified with the cleansing phase into a mixing device at a temperature above the melting point of the benefit agent after the step of admixing the benefit phase to the cleansing phase, wherein the mixing device is a static mixer unit with about 50.8 mm (2 inches) diameter including 12 mixing elements.

The benefit phase may be emulsified with the cleansing phase into a mixing device at a temperature above the melting point of the benefit agent after the step of admixing the benefit phase to the cleansing phase, at a temperature of at least about 35° C. or above about 35° C. or from about 35° C. to about 65° C., or from about 38° C. to about 45° C., wherein the mixing device is a static mixer unit with a diameter from about 25.4 mm (1 inches) to about 101.6 mm (4 inches), preferably from about 38.1 mm (1.5 inches) to about 63.5 (2.5 inches), more preferably from about 44.4 mm (1.75 inches) to about 50.8 mm (2 inches) and including from about 8 to about 20 mixing elements, preferably from about 9 to about 15 mixing elements, more from about 10 to 13 mixing elements.

Most preferably, the benefit phase may be emulsified with the cleansing phase into a mixing device at a temperature above the melting point of the benefit agent after the step of admixing the benefit phase to the cleansing phase, wherein the benefit agent comprises shea butter, at a temperature of at least about 35° C. or above about 35° C. or from about 35° C. to about 65° C., or from about 38° C. to about 45° C., wherein the mixing device is a static mixer unit with about 50.8 mm (2 inches) diameter including 12 mixing elements.

A mixing element may be defined as a component within the static mixer that promotes the mixing of fluids by inducing turbulence and creating flow patterns that enhance mixing efficiency. Suitable static mixers that can be used may be but non limiting to: SMX style static mixers from Sulzer.

The cleansing phase and the benefit phases are admixed together on the flow line downstream of the batch production line a flow rate from about 68.0 kg/min (150 lbs/min) to about 90.7 kg/min (200 lbs/min), or from about 72.6 kg/min (160 lbs/min) to about 86.2 kg/min (190 lbs/min), or from about 77.1 kg/min (170 lbs/min) to about 83.9 kg/min (185 lbs/min).

The admixing step and the subsequent emulsification step may be done at a temperature above the melting point of the benefit agent to ensure the dispersion of the benefit agent into the cleansing phase during admixing and emulsification steps. Also, the uniform temperature can help to optimize the average particle size generated through the mixing device, i.e. the static mixer as disclosed herein.

In a preferred aspect, the static mixer is positioned on the flow line just after or at proximity of a point where the cleansing phase and the benefit phases are admixed together.

In a more preferred aspect, a distance between an entrance of the static mixer and the point where the cleansing phase and the benefit phases are admixed together on the flow line may be from 101.6 mm (4 inches) to 177.8 mm (7 inches), preferably from 127.0 mm (5 inches) to 152.4 mm (6 inches)

In any aspects, the benefit agent in the personal care composition may have an average particle size from about 3 μm about 10 μm, or from about 3 μm to about 8 μm, or from about 3 μm to about 5 μm as measured according to the Particle Size Measurement as disclosed herein.

The specific benefit agent incorporation process can help to create large benefit agent particle in terms of average particle size for enhancing moisturization performance of the resulting personal care composition.

The process disclosed herein can provide personal care compositions having the benefit agent with the recited average particle sizes for achieving high deposition of the benefit agent onto skin.

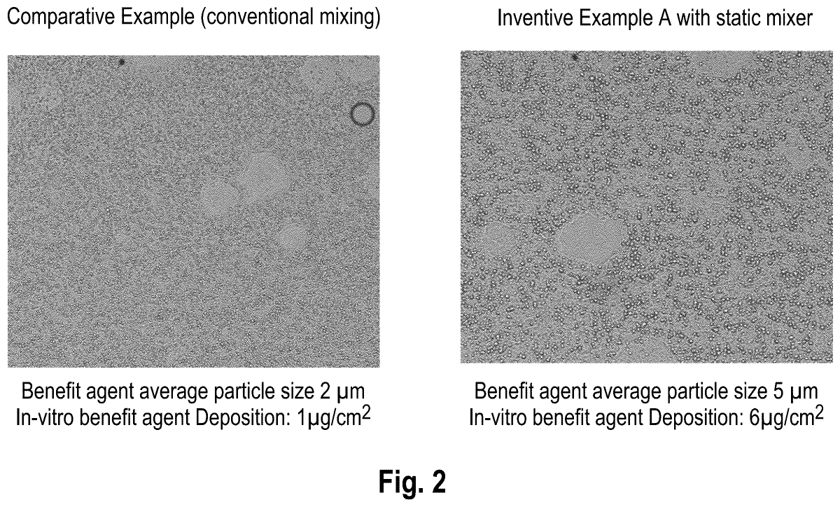

The effectiveness of the process as disclosed herein can be quantitatively assessed by measuring the deposition of the benefit agent on the skin in micrograms per square centimeter (μg/cm²). The results as shown in FIG. 2 illustrate that different configurations influence the deposition efficiency. Achieving better deposition of the benefit agents to the skin can help the benefit agents to reach their function and attributes onto skin, e.g., skin moisturization.

Hence, the personal care compositions obtained according to the process disclosed herein can provide improved skin moisturization and skin barrier function vs. personal care compositions prepared by conventional mixing methods.

The resulting personal care composition may be pumped out in a storage vessel before being transported into a package filler.

Alternatively, the resulting composition may be transported directly into a package filler, as shown in a FIG. 1 for instance.

### Benefit Agent

The personal care composition comprises a benefit phase. The benefit phase in the personal care composition may be hydrophobic or essentially anhydrous and may be substantially free of water. The benefit phase may be substantially free or free of surfactant. As a preferred aspect, the benefit phase may be anhydrous.

The benefit phase comprises a benefit agent. A benefit agent may include water-insoluble or hydrophobic benefit agent. The benefit phase comprises from about 0.1% to about 50%; preferably from about 1% to about 30%; more preferably from about 5% to about 30%, by weight of the personal care composition, of a benefit agent.

Alternatively, the personal care composition comprises from about 0.1 wt. % to about 50 wt. % of the benefit agent; preferably from about 0.5 wt. % to about 15 wt. % of the benefit agent; more preferably from about 1 wt. % to about 10 wt. % of the benefit agent; most preferably from about 2 wt. % to about 10 wt. % of the benefit agent.

The personal care composition may comprise a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 500 Pa·s to about 7750 Pa·s as measured according to the Carreau Zero Shear Viscosity Method as disclosed herein.

Alternatively, the personal care composition comprises from about 0.1 wt. % to about 50 wt. % of the benefit agent; preferably from about 0.5 wt. % to about 15 wt. % of the benefit agent; more preferably from about 10 wt. % to about 15 wt. % of the benefit agent.

The personal care composition may comprise a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 1 500 Pa·s to about 16 000 Pas as measured according to the Carreau Zero Shear Viscosity Method as disclosed herein.

The hydrophobic skin benefit agent for use in the benefit phase of the composition may have a Vaughan Solubility Parameter (VSP) of from about 5 to about 15, preferably from about 5 to less than 10. These solubility parameters are well known in the formulation arts, and are defined by Vaughan in Cosmetics and Toiletries, Vol. 103, p. 47-69, October 1988.

The benefit agent may be a natural emollient or a derivative thereof. A natural emollient is a substance derived from natural sources, such as plants or animals, that is used to moisturize, soften, and soothe the skin. Emollients form a protective barrier on the skin's surface, preventing moisture loss and helping to maintain the skin's hydration levels. Natural emollients often contain beneficial compounds like fatty acids, vitamins, and antioxidants that can nourish and improve the overall health and appearance of the skin. Examples of natural emollients include plant oils (e.g., coconut oil, olive oil), shea butter, cocoa butter, and beeswax, preferably shea butter. A derivative of a natural emollient may be a natural emollient oil.

The benefit agent may be selected from the group consisting of lanolin; derivatives of lanolin; natural waxes; natural triglycerides; synthetic triglycerides; and mixtures thereof.

Non-limiting examples of lanolin and lanolin derivatives suitable for use as hydrophobic skin benefit agents herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol ricinoleate, and mixtures thereof.

Alternatively, non-limiting examples glycerides suitable for use as hydrophobic skin benefit agents herein include castor oil, soybean oil, derivatized soybean oils such as maleated soybean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, shea butter, and mixtures thereof.

The benefit agent may be selected from argan oil, castor oil, soybean oil, derivatized soybean oils, maleated soybean oil, safflower oil, cotton seed oil, corn oil, walnut oil, peanut oil, olive oil, cod liver oil, sweet almond oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, shea butter, groundnut oil, camellia oil, beauty-leaf oil, rapeseed oil, coconut kernel, coriander oil, marrow oil, wheat germ oil, jojoba oil or liquid jojoba wax, linseed oil, macadamia oil, corn germ oil, hazelnut oil, walnut oil, vemonia oil, apricot kernel oil, olive oil, evening-primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil; or alternatively caprylic/capric acid triglycerides and mixtures thereof.

In a preferred aspect, the benefit agent may be selected from argan oil, castor oil, soybean oil, maleated soybean oil, avocado oil, coconut oil, jojoba oil, cocoa butter, shea butter, and mixtures thereof.

In a more preferred aspect, the benefit agent may be selected from argan oil, soybean oil, maleated soybean oil, shea butter, and mixtures thereof.

In an even more preferred aspect, the benefit agent may comprise soybean oil or shea butter. In that aspect, the personal care composition may comprise from about 0.5 wt. % to about 15 wt. % of soybean oil or shea butter; preferably from about 1 wt. % to about 10 wt. % of soybean oil or shea butter, most preferably from about 2 wt. % to about 10 wt. % of soybean oil or shea butter.

The benefit phase may comprise a hydrophobic benefit agent and optionally a lipid bilayer structurant. The lipid bilayer structurant may comprise glyceryl monooleate, glyceryl monostearate, glyceryl monolaurate, or a mixture thereof. The benefit agent may comprise argan oil, soybean oil, maleated soybean oil, shea butter, or a mixture thereof.

In a most preferred aspect, a process for manufacturing a personal care composition comprising a cleansing phase and a benefit phase, wherein the composition is substantially free of alkyl sulfate and alkyl ether sulfate type of surfactants is provided and comprises the steps of, preferably in that order:
(a) forming the cleansing phase of the personal care composition in a main mixing vessel on a batch production line; wherein the cleansing phase is defined as an aqueous structured surfactant phase;
wherein the cleansing phase comprises:
   (a1) a N-acyl alaninate surfactant, preferably from about 5 wt. % to about 20 wt. % of sodium cocoyl alaninate; preferably from about 7 wt. % to about 16 wt. % of sodium cocoyl alaninate; more preferably from about 9 wt. % to about 13 wt. % of sodium cocoyl alaninate; most preferably from about 10 wt. % to about 12 wt. % of sodium cocoyl alaninate;
   (a2) a zwitterionic or amphoteric surfactant, preferably a zwitterionic surfactant comprising cocamidopropyl betaine, more preferably from about 0.01 wt. % to about 20 wt. % of cocamidopropyl betaine; preferably from about 0.1 wt. % to about 10 wt. % of cocamidopropyl betaine; more preferably from about 1 wt. % to about 10 wt. % of cocamidopropyl betaine; most preferably from about 2 wt. % to about 5 wt. % of cocamidopropyl betaine; and
   (a3) a structuring system comprising from about 0.5 wt. % to about 5 wt. % of an emulsifying agent; and from about 0.01 wt. % to about 10 wt. % of a rheology modifier;
   wherein the emulsifying agent comprises a glyceryl ester, preferably from about 1 wt. % to about 3 wt. % of the glyceryl ester; more preferably from about 1 wt. % to about 2.75 wt. % of the glyceryl ester; most preferably from about 1 wt. % to about 2.5 wt. % of the glyceryl ester, wherein the glyceryl ester is selected from glyceryl laurate, glyceryl caprate, glyceryl caprylate, glyceryl caprylate/caprate, glyceryl stearate, and a mixture thereof, preferably glyceryl caprylate/caprate;
   wherein the rheology modifier comprises a hydrophobically modified polysaccharide, especially a modified starch, wherein the modified starch is selected from the group consisting of hydroxypropyl starch phosphate, distarch phosphate, sodium carboxymethyl starch, and mixtures thereof, preferably hydroxypropyl starch phosphate, from about 0.01 wt. % to about 10 wt. % of hydroxypropyl starch phosphate, preferably from about 0.1 wt. % to about 5 wt. % of hydroxypropyl starch phosphate, more preferably from about 0.5 wt. % to about 1.5 wt. % of hydroxypropyl starch phosphate, most preferably from about 0.6 wt. % to about 1.0 wt. % of hydroxypropyl starch phosphate;
(b) admixing the benefit phase to the cleansing phase on a flow line downstream of the batch production line, wherein the benefit phase comprises from about 0.1 wt. % to about 50 wt. % of a benefit agent, from about 0.5 wt. % to about 15 wt. % of shea butter; preferably from about 1 wt. % to about 10 wt. % of shea butter, most preferably from about 2 wt. % to about 10 wt. % of soybean shea butter; and
(c) recovering the resultant personal care composition.

The step of forming the cleansing phase may comprise the following steps, preferably in that order:
adding water;
adding the rheology modifier under a high shear or a turbine agitation;
adding the zwitterionic or amphoteric surfactant;
optionally adding a cationic deposition polymer;
adding the anionic surfactant being substantially free of sulfate, preferably the N-acyl amino acid surfactant, most preferably the N-acyl alaninate surfactant;
adding the emulsifying agent directly to the main mixing vessel or from a first jacketed vessel to the main mixing vessel;
adding a preservative; and
adjusting the pH such that the personal care composition has a pH of from about 4.0 to about 5.5.

Alternatively, the step of forming the cleansing phase may comprise the following steps, preferably in that order:
adding water;
adding cocamidopropylbetaine;
optionally adding a cationic deposition polymer;
optionally adding a preservative;
adding the emulsifying agent directly to the main mixing vessel or from a first jacketed vessel to the main mixing vessel;
adding the rheology modifier under a high shear or a turbine agitation;
adding N-acyl alaninate surfactant; and
adjusting the pH such that the personal care composition has a pH of from about 4.0 to about 5.5.

The emulsifying agent may comprise a glyceryl ester, wherein the glyceryl ester is melted in the first jacketed vessel at a temperature above a melting point of the glyceryl ester, more preferably from about 30° C. to about 70° C., or from about 38° C. to about 65° C., most preferably from about 40° C. to about 63° C.

The personal care composition may comprise from about 0.3 wt. % to about 1.5 wt. % of hydroxypropyl starch phosphate and from about 0.1 wt. % to about 0.5 wt. % of xanthan gum, preferably from about 0.3 wt. % to about 1.0 wt. % of hydroxypropyl starch phosphate and from about 0.1 wt. % to about 0.4 wt. % of xanthan gum, more preferably from about 0.75 wt. % to about 0.90 wt. % of hydroxypropyl starch phosphate and from about 0.2 wt. % to about 0.3 wt. % of xanthan gum.

In that aspect, hydroxypropyl starch phosphate may be added to the main mixing vessel under a high shear or a turbine agitation. Xanthan gum may be added as a slurry with the emulsifying agent in the first jacketed vessel, preferably wherein the emulsifying agent comprises glyceryl caprylate/caprate.

In that aspect, the emulsifying agent may comprise glyceryl caprylate/caprate, wherein glyceryl caprylate/caprate is melted in the first jacketed scrape wall vessel at a temperature above a melting point of glyceryl caprylate/caprate, more preferably from about 35° C. to about 50° C., or from about 38° C. to about 45° C., most preferably from about 38° C. to about 42° C.

The benefit phase may be provided from a second jacketed vessel and the benefit agent is melted prior to admix the benefit phase to the cleansing phase on the flow line downstream of the batch production line at a temperature of at least about 35° C. or above about 35° C. or from about 35° C. to about 65° C., or from about 38° C. to about 45° C.

The benefit phase may be admixed to the cleansing phase at a temperature above a melting point of the benefit agent, preferably at a temperature of at least about 35° C. or above about 35° C. or from about 35° C. to about 65° C., or from about 38° C. to about 45° C.

The benefit phase may be emulsified with the cleansing phase into a mixing device at a temperature above the melting point of the benefit agent, at a temperature of at least about 35° C. or above about 35° C. or from about 35° C. to about 65° C., or from about 38° C. to about 45° C., wherein the mixing device is a static mixer unit with a diameter from about 25.4 mm (1 inches) to about 63.5 mm (2.5 inches), preferably from about 38.1 mm (1.5 inches) to about 57.1 (2.25 inches), more preferably from about 44.4 mm (1.75 inches) to about 50.8 mm (2 inches) and including from about 8 to about 20 mixing elements, preferably from about 9 to about 15 mixing elements, more from about 10 to 13 mixing elements.

Most preferably, the benefit phase may be emulsified with the cleansing phase into a mixing device at a temperature above the melting point of the benefit agent, wherein the benefit agent comprises shea butter, at a temperature of at least about 35° C. or above about 35° C. or from about 35° C. to about 65° C., or from about 38° C. to about 45° C., wherein the mixing device is a static mixer unit with about 50.8 mm (2 inches) diameter including 12 mixing elements.

The benefit agent in the personal care composition may have an average particle size from about 3 μm about 10 μm, or from about 3 μm to about 8 μm, or from about 3 μm to about 5 μm as measured according to the Particle Size Measurement as disclosed herein.

### Optional Ingredients

As can be appreciated, the compositions described herein may include a variety of optional components to tailor the properties and characteristics of the composition. As can be appreciated, suitable optional components are well known and can generally include any components which are physically and chemically compatible with the essential components of the compositions described herein. Optional components should not otherwise unduly impair product stability, aesthetics, or performance. Individual concentrations of optional components can generally range from about 0.001% to about 10%, by weight of the composition. Optional components can be further limited to components which will not impair the clarity of a translucent composition.

Still, the personal care composition may not include or may be free of direct dyes, oxidative dyes, parabens, or mixtures thereof.

Optional components may include, but are not limited to dyes, pigments, humectants, conditioning agents, skin exfoliating agents, anti-dandruff actives, and chelating agents. Additional suitable optional ingredients include but are not limited to particles, anti-microbials, foam boosters, anti-static agents, moisturizing agents, propellants, self-foaming agents, pearlescent agents, opacifiers, sensates, suspending agents, solvents, diluents, anti-oxidants, vitamins, and mixtures thereof.

### Physical Contact Between the Cleansing and Benefit Phases

In the personal care composition, the cleansing phase and the benefit phase may be in physical contact. The phases may be blended or mixed to a significant degree, but still be physically distinct such that the physical distinctiveness is undetectable to the naked eye.

The phases can also be made to occupy separate and distinct physical spaces inside a package in which the phases can be stored. In such an arrangement, the structured cleansing phase and the benefit phase can be stored such that the phases are not in direct contact with one another.

Alternatively, the personal care composition may be a multiphase personal care composition. In that aspect, the phases of the personal care composition may be made to occupy separate but distinct physical spaces inside the package in which they are stored, but are in direct contact with one another (i.e., they are not separated by a barrier and they are not emulsified or mixed to any significant degree).

The cleansing phase and the benefit phase can be in physical contact while remaining visibly distinct to give, for example, a striped or marbled or geometric configuration.

### Forms and Uses

### Product Form

The personal care composition may be presented in typical personal care formulations. They may be in the form of solutions, dispersion, emulsions, foams, and other delivery mechanisms. The personal care composition may be a rinse-off composition.

The personal care composition may be extrudable or dispensable from a single chamber package. The personal care compositions can be in the form of liquid, semi-liquid, cream, lotion or gel, or solid compositions intended for topical application to skin.

Examples of personal care compositions, preferably personal cleansing compositions can include but are not limited to body wash, moisturizing body wash, foaming body wash, shower gels, a shower or bath cream, skin cleansers, cleansing milks, body wash, in shower body moisturizer, gel, emulsion, oil, mousse or spray.

The personal care composition may not be in the form of a liquid hand wash or a liquid hand sanitizer.

The product forms contemplated for purposes of defining the personal care compositions and methods are rinse-off formulations by which it is meant that the product is applied topically to the skin and then subsequently (i.e., within minutes) rinsed away with water, or otherwise wiped off using a substrate or other suitable removal means.

### Uses

The personal care composition as set out hereinabove may be used for improving the lather of the composition.

The personal care composition as set out hereinabove may be used for suspending benefits agents

The personal care composition can advantageously provide relatively improved ecotoxic or ecologically friendly environmental profile.

The personal care composition can help to provide good aesthetic properties such as good foam, and is thick and creamy in texture, is silky to the touch and affords conditioning.

### Test Methods

It is understood that the test methods that are disclosed in the Test Methods Section of the present application should be used to determine the respective values of the parameters of the personal care compositions described and claimed herein.

### Cylinder Method

Lather can be measured in accordance with the Cylinder Method. Lather volume is measured using a graduated cylinder and a rotating mechanical apparatus. A 1,000 ml graduated cylinder is used which is marked in 10 ml increments, has a height of 14.5 inches at the 1,000 ml mark from the inside of its base, and has a neck at its top fitted for a plastic insert cap (for example, Pyrex No. 2982). Moderately hard water (about 7 gpg or about 120 ppm) is prepared by dissolving 1.14 grams calcium chloride dihydrate and 1.73 grams magnesium chloride hexahydrate into one U.S. gallon distilled water. The water is maintained at between 40.5-43.3° C. (105-110° F.). The graduated cylinder is heated to about the same temperature by flushing with excess tap water at the same temperature for about 15 seconds, then drying its exterior and shaking briefly upside down to dry the interior. 100.0 grams of the moderately hard water at the indicated temperature is weighed directly into the graduated cylinder. The cylinder is clamped in a mechanical rotating device, which clamps the cylinder vertically with an axis of rotation that transects the center of the graduated cylinder. Using a 3- or 4-place metric balance, invert the plastic cap for the graduated cylinder onto the balance pan and weigh 0.500 grams of composition for compositions less than 19% surfactant (weigh 0.250 grams of composition for compositions greater or equal than 19% surfactant) to within 4 milligrams accuracy, using a holder to keep the cap level. Insert the cap into the graduated cylinder neck while being careful that all composition is now in the space in the cylinder interior. For compositions with very low viscosity which will not remain on the cap surface, 500 mg composition can be added directly to the graduated cylinder. Rotate the cylinder for 25 complete revolutions at a rate of about 10 revolutions per 18 seconds to create a lather and stop in a level, vertical position. When the cylinder stops in a vertical position, start a digital stopwatch. Observing the water draining at the bottom, record the time to the nearest second when the water height measures 50 cc, then 60 cc, then 70 cc and so on until at least 90 cc has drained. Measure and record the total height of the foam in the column interior, which is the lather volume. If the top surface of the lather is uneven, the lowest height at which it is possible to see halfway across the graduated cylinder is the lather volume (ml). If the lather is coarse such that a single or only a few foam cells (“bubbles”) reach across the entire cylinder, the height at which at least about 10 foam cells are required to fill the space is the lather volume, also in ml up from the base. When measuring the lather height, bubbles that are larger than about 25.4 mm (1 inch) across at the top surface are considered free air and not lather. The measurement is repeated and at least three results averaged to obtain the lather volume. In a spreadsheet, calculate the lather density at each observed time point as the volume of foam (total height minus water height) divided by the weight of the foam (100.5 grams minus the weight of water observed, using a density of 1.00 g/cc for water). Fit the 3 time points closest to (ideally, also bracketing) 20 seconds to a 2^{nd} order polynomial equation. Solve the equation for the lather density at 20 seconds, which is the lather density of the composition. Multiply the lather volume by the lather density to obtain the lather mass, in grams.

The entire measurement process should take less than about 3 minutes in order to maintain the desired temperature.

The personal care composition may comprise a lather volume from about 375 mL to about 575 mL, preferably from about 395 mL to about 560 mL, more preferably from about 400 mL to about 545 mL, most preferably from about 450 mL to about 540 mL as measured according to the Cylinder Method as disclosed herein.

### Carreau Zero Shear Viscosity Method

The Carreau Zero Shear Viscosity of a material which is a phase or a composition of the personal care composition, can be measured either prior to combining in the composition, after preparing a composition, or first separating a phase or component from a composition by suitable physical separation means, such as centrifugation, pipetting, cutting away mechanically, rinsing, filtering, or other separation means.

A controlled stress rheometer such as a TA Instruments Discovery HR2 Rheometer is used to determine the Carreau Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First the material (i.e. the sample to be tested) is positioned on the rheometer base plate, the measurement geometry (upper plate) is moved into position 1.1 mm above the base plate. Excess material at the geometry edge is removed by scraping after locking the geometry. The geometry is then moved to the target 1 mm position above the base plate and a pause of about 1 minute is allowed to allow loading stresses to relax. This loading procedure ensures no tangential stresses are loaded at the measurement onset, which can influence results obtained. If the material comprises particles discernible to the eye or by feel (beads, e.g.) which are larger than about 150 microns in number average diameter, the gap setting between the base plate and upper plate is increased to the smaller of 4 mm or 8-fold the diameter of the 95th volume percentile particle diameter. If a phase has any particle larger than 5 mm in any dimension, the particles are removed prior to the measurement.

The measurement is performed by applying a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. If the measurement result is incomplete, for example if material is observed to flow from the gap, results obtained are evaluated with incomplete data points excluded. If there are insufficient points to obtain an accurate measurement, the measurement is repeated with an increased number of sample points.

The Carreau Zero Shear Viscosity (Pa·s) is obtained by fitting the data to a Carreau viscosity model.

The personal care composition may have a Carreau Zero Shear Viscosity from about 200 Pa·s to about 16 000 Pa·s, preferably from about 500 Pa·s to about 13 000 Pa·s, more preferably from about 1000 Pa·s to about 12000 Pa·s, even more preferably from about 2900 Pa·s to about 11775 Pa·s, most preferably from about 4500 Pa·s to about 11660 Pa·s, or from about 500 Pa·s to about 7750 Pa·s; or from about 1 500 Pa·s to about 16 000 Pa·s as measured according to the Carreau Zero Shear Viscosity Method as disclosed herein.

### Ultracentrifugation Method

The Ultracentrifugation Method is used to determine the percent of a structured domain or an opaque structured domain (e.g., a lamellar phase) that is present in a multiphase personal care composition. The method involves the separation of the composition by ultracentrifugation into separate but distinguishable layers. The multiphase personal care composition of the present disclosure can have multiple distinguishable layers (e.g. a structured surfactant layer, and a benefit layer).

A composition is separated by ultracentrifuge into separate but distinguishable layers.

First, dispense about 4 grams of composition into a Beckman Centrifuge Tube (11×60 mm) to fill the tube. Place the centrifuge tubes in an ultracentrifuge (Beckman Model L8-M or equivalent) using a sling rotor and ultracentrifuge using the following conditions: 50,000 rpm, 24 hours, and 40° C.

Measure the relative phase volumes of the phases the composition by measuring the height of each layer using an Electronic Digital Caliper (within 0.01 mm). Layers are identified by those skilled in the art by physical observation techniques paired with chemical identification if needed. For example, the structured surfactant layer is identified by transmission electron microscopically (TEM), polarized light microscopy, and/or X-ray diffraction for the present disclosure as a structured lamellar phase comprising multiple lamellar vesicles, and the hydrophobic benefit layer is identified by its low moisture content (less than 10% water as measured by Karl Fischer Titration). The total height Hₐ is measured which includes all materials in the ultracentrifuge tube. Next, the height of each layer is measured from the bottom of the centrifuge tube to the top of the layer, and the span of each layer algebraically determined by subtraction. The benefit layer may comprise several layers if the benefit phase has more than one component which may phase splits into liquid and waxy layers, or if there is more than one benefit component. If the benefit phase splits, the sum of the benefit layers measured is the benefit layer height, H_{b}. In some cases, in case of incomplete separation of the benefit phase from the cleansing phase, the resulting emulsion phase is considered to be part of the benefit phase and is included in the measurement of the benefit layer height, H_{b}. Generally, a hydrophobic benefit layer when present, is at the top of the centrifuge tube.

The cleansing phase may comprise several layers or a single layer, H_{c}. There may also be a micellar, unstructured, clear isotropic layer which may contain the rheology modifiers at the bottom or next to the bottom of the ultracentrifuge tube. The layers immediately above the isotropic phase generally comprise higher surfactant concentration with higher ordered structures (such as liquid crystals). These structured layers are sometimes opaque to naked eyes, or translucent, or clear. There may be several structured layers present, in which case H_{c} is the sum of the individual structured layers. If any type of polymer-surfactant phase is present, it is considered a structured phase and included in the measurement of H_{c}. The sum of the aqueous phases is Hₛ.

Finally, the structured domain volume ratio is calculated as follows:

$$Structured Domain Volume Ratio = {H}_{c} / {H}_{s} * 100 \%$$

If there is no benefit phase present, use the total height as the surfactant layer height, Hₛ=Hₐ. The Structured Domain Volume Ratio is the Lamellar Phase %.

The personal care composition may have a Structured Domain Volume Ratio of at least about 40%, alternatively at least about 45%, alternatively at least about 50%, alternatively at least about 55%, alternatively at least about 60%, alternatively at least about 65%, alternatively at least about 70%, alternatively at least about 75%, alternatively at least about 80%, alternatively at least about 85%, and alternatively greater than about 90% by volume of the aqueous structured surfactant phase.

### Carbon-Chain Length Distribution Test Method for a N-Acyl Alaninate Surfactant

The samples to be tested are diluted to about 0.6% (w/w) in a volatile solvent such as a mixture of chloroform and methanol. A 100 μL aliquot of this sample stock solution is transferred to a vial and the solvent is dried under nitrogen. A 0.6% (w/w) internal standard stock solution is prepared separately by diluting tridecanoic acid in an aprotic solvent such as pyridine. A 100 μL aliquot of internal standard stock is added to the dried sample vial with an excess of 300 μL O-Bis(trimethylsilyl)trifluoroacetamide with 1% Trimethylchlorosilane (BSTFA-TMCS) to derivatize the N-acyl alaninate surfactant and the internal standard.

A retention time standard has been prepared in a similar way to the surfactant samples. The retention time standard used was lauroyl alanine, which is commercially available. All prepared vials are sealed and either heated at 90° C. for one hour or kept at room temperature for at least 12 hours for derivatization to complete.

### Chromatographic System

The gas chromatograph is equipped with a flame-ionization detector. The derivatized samples are injected into a 25:1 split inlet and onto an Agilent DB-1 2.5 m×0.25 mm×0.25 μm capillary column that has been cut from a commercial length column. The H₂ carrier gas is in constant flow mode of 1.5 mL/min. The oven temperature program [40° C., ramped (20° C./min) to 100° C., ramped (40° C./min) to 350° C. (7 min)] has a total run time of 16.25 minutes. The flame ionization detection (FID) temperature is 350° C. with 30 mL/min H₂ flow, 400 mL/min Air flow, and 25 mL/min Makeup (N₂) flow. Peaks are identified based on the retention times of the standards.

Peak areas are used to determine the relative carbon-chain length distribution of the acyl N-alaninate surfactant.

### Particle Size Measurement

The number weighted average particle size of the benefit agent is measured in neat product under a differential interference contrast optical microscope with a 10× objective lens. The particle size distribution is counted manually. All particles of the benefit agent are assumed as uniform spheres in this disclosure. For irregular shaped particles, the longest axis is used as the diameter for the particle size distribution counting. The number weighted average of all particles of the benefit agent is defined as the average particle size of the benefit agent.

### In-Vitro Deposition Method

### In-Vitro Deposition Evaluation Method:

The In-vitro Deposition Evaluation Method measures the deposition of benefit agents on a skin mimic. The method compares the quantity of benefit agent of the skin mimic surface before and after cleansing in an automated cleansing unit, such as the automated cleansing unit described in co-pending and co-assigned Multiphase Personal Care Composition With Enhanced Deposition, U.S. application Ser. No. 12/510,880 (filed Jul. 28, 2009) and In-Vitro Deposition Evaluation Method for Identifying Personal Care Compositions Which Provide Improved Deposition of Benefit Agents, U.S. application Ser. No. 12/511,034 (filed Jul. 28, 2009).

The In-vitro Deposition Evaluation Method uses two 12-well plates (hereinafter referred to as “plates”). Suitable 12-well plates are commercially available from Greiner bio-one. For example, the Cellstar® 12 well suspension culture plate has 3 rows and 4 columns with a well volume of about 6.2 mL. The Cellstar® 12 well suspension culture plate comprises the approximate dimensions of 19 mm in height, 127 mm in length and 85 mm in width. The Cellstar® 12 well suspension culture plate has a well diameter of 23 mm, a well depth of 15 and a well to well spacing of 2 mm. A Cellstar® 12 well suspension culture plate is provided for containing the samples comprising the personal care composition in the Examples above.

The In-vitro Deposition Evaluation Method uses approximately 120 g of bodies for two plates. Five grams of bodies carefully loaded into each of the 12 wells of the two plates to ensure the same quantity is loaded into each well. Each body is a spherical stainless steel bearing that is approximately 2 mm in circumference. Each body comprises ferrometallic material. Suitable bodies are those available from WLB Antriebeselemente Gmbh, Scarrastrasse 12, D-68307 Mannheim, Germany.

### EXAMPLES

The following examples further describe the process for manufacturing the personal care compositions and the personal care compositions described herein. The examples are given solely for the purpose of illustration and are not to be construed as limitations of the present disclosure, as many variations thereof are possible without departing from the spirit and scope of the disclosure. Where applicable, ingredients are identified by chemical or CTFA name, or otherwise defined below.

### Process for Manufacturing the Personal Care Composition—Example A

An Example A according to the process A disclosed herein is made as follow:

The order of addition is as follow in a main mixing vessel on a batch production line: water, hydroxypropyl starch phosphate a turbine agitation with an off-center pitch blade turbine, following by cocamidopropyl betaine, and guar hydroxypropyltrimonium chloride. Then, sodium cocoyl alaninate is added to the main mixing vessel over two minutes with a total mixing time of 6 minutes. After mixing for a minimum of 3 minutes, glyceryl caprylate/caprate is premelted in a first jacketed vessel at a temperature of 38-45° C. A slurry of xanthan gum within the premelted glyceryl caprylate/caprate is added to the main mixing vessel. Then, sodium benzoate and sodium salicylate are added.

Upon completion of addition and mixing step, a 25% citric acid premix is added to the main mixing vessel over a 1 minute period to adjust the pH to about 5.0. The agitation of the main mixing vessel is adjusted with a turbine agitation to 35-45 rpm. A perfume is added over a 5 minute mix step. The product was kept mixed until homogeneous. Prior to the admixing step, all agitation in the main mixing vessel are turned off.

A benefit phase comprising shea butter as a benefit agent is placed into a separate second jacketed scrape wall tank and heated to between 40° C. and 43° C.

On a flow line downstream of the batch production line, the benefit phase is admixed to the cleansing phase between 40° C. and 43° C.

The benefit phase is then emulsified with the cleansing phase between 40° C. and 43° C. into a static mixer unit with a diameter of about 50.8 mm (2 inches) and including about 12 mixing elements. The cleansing phase and the benefit phases are mixed together on the flow line downstream of the batch production line a flow rate of about 78.0-81.6 kg/min (172-180 lbs/min).

### Process for Manufacturing the Personal Care Composition—Example B

An Example B according to the process B disclosed herein is made as follow:

The order of addition is as follow in a main mixing vessel on a batch production line: water, cocamidopropyl betaine, optionally guar hydroxypropyltrimonium chloride, sodium benzoate and sodium salicylate. After mixing for a minimum of 3 minutes, glyceryl caprylate/caprate is premelted in a first jacketed vessel at a temperature of 38-45° C. A slurry of xanthan gum within the premelted glyceryl caprylate is added to the main mixing vessel. When xanthan gum is not used, the premelted glyceryl caprylate is added to the main mixing vessel.

After adequate mixing times, hydroxypropyl starch phosphate or any rheology modifier is added to the main mixing vessel with a high shear or a turbine agitation. Sodium cocoyl alaninate is added to the main mixing vessel over two minutes with a total mixing time of 6 minutes. Upon completion of addition and mixing step, a 25% citric acid premix is added to the main mixing vessel over a 1 minute period to adjust the pH to about 5.0. The agitation of the main mixing vessel is adjusted with a turbine agitation to 35-45 rpm and side sweeps to 10-14 rpm. A perfume is added over a 5 minute mix step. The product was kept mixed until homogeneous. Prior to the admixing step, all agitation in the main mixing vessel are turned off.

A benefit phase comprising shea butter as a benefit agent is placed into a separate second jacketed scrape wall tank and heated to between 40° C. and 43° C.

On a flow line downstream of the batch production line, the benefit phase is admixed to the cleansing phase between 40° C. and 43° C.

The benefit phase is then emulsified with the cleansing phase between 40° C. and 43° C. into a static mixer unit with a diameter of about 50.8 mm (2 inches) and including about 12 mixing elements. The cleansing phase and the benefit phases are mixed together on the flow line downstream of the batch production line a flow rate of about 78.0-1.6 kg/min (172-180 lbs/min).

A sample process diagram for a manufacturing method is shown in FIG. 1.

A Comparative Example is prepared with the same ingredients and proportions of Example A.

The Comparative Example was prepared by the following comparative process: adding water in a main mixing vessel. Then, the following ingredients were added in that order with continuously mixing until obtaining a homogenous mixture: cocamidopropyl betaine, optionally guar hydroxypropyltrimonium chloride, sodium benzoate, and sodium salicylate. In a separate vessel, glyceryl caprylate/caprate was heated to about 50° C. to melt and the premelted glyceryl caprylate/caprate was added to the main mixing vessel. Then, the following ingredients were added in the main mixing vessel with continuously mixing: sodium cocoyl alaninate, glyceryl caprylate/caprate, xanthan gum, and hydroxypropyl starch phosphate. The pH was adjusted by adding citric acid solution (50% active) to pH=5.0±0.2. Then, perfume has been added. The product was kept mixed until homogeneous. Shea butter was heated to about 50° C. Then, the melted shea butter was added into the main mixing vessel with continuously mixing until homogeneous. The benefit phase was directly mixed to the cleansing phase in the main mixing vessel.

FIG. 2 provides two images of the Comparative Example made by conventional mixing and the Example A according to the present disclosure, both as neat products under a differential interference contrast optical microscope with a 10× objective lens.

In the Example A of the present disclosure versus the comparative example, the admixing step on the flow line downstream the batch production line, the use of the static mixer and the uniform temperature of the benefit phase from the admixing step to the emulsification step could lead to a personal care composition wherein the benefit agent in the personal care composition has an average particle size of 5 μm as measured according to the Particle Size Measurement as disclosed herein versus an average particle size of 2 μm for the Comparative Example.

Also, the in-vitro benefit agent deposition is 6 μg/cm² for the Example A compared to a lower deposition of 1 μg/cm² for the Comparative Example as measured according to the In-vitro Deposition Evaluation Method.

Hence, the benefit agent in the personal care composition may have an average particle size from about 3 μm about 10 μm, or from about 3 μm to about 8 μm, or from about 3 μm to about 5 μm as measured according to the Particle Size Measurement as disclosed herein.

The following illustrative examples can be made according to the process disclosed herein.

| Ingredients (wt. %) | Ex. 1 | Ex. 2 | Ex. 3 | Ex. 4 | Ex. 5 | Ex. 6 |
|---|---|---|---|---|---|---|
| Sodium cocoyl alaninate | 14.0 | 14.0 | 14.0 | 14.0 | 14.0 | 14.0 |
| (low salt)¹ | | | | | | |
| Cocamidopropyl betaine² | 4.0 | 4.0 | 4.0 | 4.0 | 4.0 | 4.0 |
| Glyceryl | 2.0 | — | 1.0 | 2.0 | 2.0 | 2.0 |
| caprylate/caprate³ | | | | | | |
| Hydroxypropyl starch | 1.0 | 1.0 | 1.0 | 1.0 | 1.0 | — |
| phosphate⁸ | | | | | | |
| Trideceth-3¹⁰ | — | 2.0 | — | — | — | — |
| Acrylates/C10-C30 alkyl | — | — | — | — | — | 0.1 |
| acrylates crosspolymer¹¹ | | | | | | |
| (Aqupec SER-300) | | | | | | |
| Xanthan gum⁹ | — | — | 0.3 | — | — | — |
| Argan oil¹² | — | — | — | 1.94 | — | — |
| Shea Butter¹³ | — | — | — | — | 1.94 | — |
| Soybean oil⁴ | 1.94 | 1.94 | 1.94 | — | — | 1.94 |
| Glyceryl Monooleate | 0.02 | 0.02 | 0.02 | 0.02 | 0.02 | 0.02 |
| BHT | 0.04 | 0.04 | 0.04 | 0.04 | 0.04 | 0.04 |
| Citric Acid⁵ | Adjust | Adjust | Adjust | Adjust | Adjust | Adjust |
| | pH 5 | pH 5.0 | pH 5 | pH 5 | pH 5 | pH 5 |
| Sodium Benzoate⁶ | 0.45 | 0.45 | 0.45 | 0.45 | 0.45 | 0.45 |
| Sodium salicylate⁷ | 0.40 | 0.40 | 0.40 | 0.40 | 0.40 | 0.40 |
| Perfume | 1.0 | 1.0 | 1.0 | 1.0 | 1.0 | 1.0 |
| Water | q.s. | q.s. | q.s. | q.s. | q.s. | q.s. |
| Formula total | 100 | 100 | 100 | 100 | 100 | 100 |
| Ratio alaninate/GCC or | 7 | 7 | 14 | 7.0 | 7.0 | 7.0 |
| nonionic | | | | | | |
| Cylinder lather volume | 493 | 395 | 510 | 500 | 465 | 435 |
| (avg All) (mL) | | | | | | |
| Carreau Zero shear | 4822 | 7502 | 7280 | 4204 | 4336 | 3759 |
| Viscosity (Pa · s) | | | | | | |
| Lamellar Phase Volume | 79% | 78% | 71% | 80% | 80% | 83% |
| (%) | | | | | | |

| Ingredients (wt. %) | Ex. 7 | Ex. 8 | Ex. 9 | Ex. 10 |
|---|---|---|---|---|
| Sodium cocoyl alaninate | 14.0 | 14.0 | 14.0 | 14.0 |
| (low salt)¹ | | | | |
| Cocamidopropyl betaine² | 4.0 | 4.0 | 4.0 | 4.0 |
| Glyceryl laurate¹⁴ | 1.0 | 1.0 | — | — |
| Glyceryl stearate¹⁵ | — | — | 1.0 | 1.0 |
| Hydroxypropyl starch phosphate⁸ | 1.0 | 1.0 | 1.0 | 1.0 |
| Soybean oil⁴ | 1.94 | 9.70 | 1.94 | 9.70 |
| Glyceryl Monooleate | 0.02 | 0.10 | 0.02 | 0.10 |
| BHT | 0.04 | 0.20 | 0.04 | 0.20 |
| Citric Acid⁵ | Adjust | Adjust | Adjust | Adjust |
| | pH 5.0 | pH 5.0 | pH 5.0 | pH 5.0 |
| Sodium Benzoate⁶ | 0.45 | 0.45 | 0.45 | 0.45 |
| Sodium salicylate⁷ | 0.40 | 0.40 | 0.40 | 0.40 |
| Perfume | 1.0 | 1.0 | 1.0 | 1.0 |
| Water | q.s. | q.s. | q.s. | q.s. |
| Formula total | 100 | 100 | 100 | 100 |
| Cylinder lather volume (avg All) (mL) | 540 | 510 | 510 | 490 |
| Carreau Zero shear Viscosity (Pa · s) | 3720 | 4039 | 4666 | 4910 |
| Lamellar Phase Volume (%) | 82% | 78% | 77% | 76% |

Definitions of Components
*¹Sodium cocoyl alaninate; Eversoft ACS (contains circa 1 wt. % NaCl), Supplier Sino Lion;
*²Cocamidopropyl Betaine; SensaFoam ™ CK PH 12/MB, Supplier Kensing ™, having a 30% active matter and a sodium chloride content of about 5 wt. %.
*³Glyceryl caprylate/caprate; Stepan Mild GCC, Supplier Stepan, containing mono, di, triglycerides, Supplier Stepan Company;
*⁴Soybean oil, RBD Soybean oil, Supplier Cargill
*⁵Citric acid powder; Supplier Yixing Union Biochemical
*⁶Sodium benzoate; Supplier Wuhan Youji Industries
*⁷Sodium salicylate; Supplier JQC Huayn Pharmaceutical Co Ltd.
*⁸Hydroxypropyl starch phosphate, Structure XL, Supplier Nouryon Chemicals
*9Xanthan gum, Keltrol 1000, Supplier CPKelco
*¹⁰Trideceth-3, Iconal TDA-3, Supplier BASF Corp.
*¹¹Acrylates/C10-C30 alkyl acrylates crosspolymer, Aqupec SER-300, Supplier Sumitomo Seika Chemicals
*¹²Argan oil, Argania Spinosa Kernel Oil, Supplier Carrubba
*¹³Shea Butter, Shebu refined, Supplier Rita
*¹⁴Glyceryl laurate, Monomuls ® 90-L 12, Supplier BASF
*¹⁵Glyceyl stearate, Lexemul ® 515 MB, Supplier Inolex
q.s.: sufficient quantity

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for manufacturing a personal care composition, the process comprising a cleansing phase and a benefit phase, wherein the composition is substantially free of alkyl sulfate and alkyl ether sulfate type of surfactants, wherein the process comprises the steps of, in that order:
(a) forming the cleansing phase of the personal care composition in a main mixing vessel on a batch production line; the cleansing phase being an aqueous structured surfactant phase;
wherein the cleansing phase comprises:
(a1) an anionic surfactant being substantially free of sulfate;
(a2) a zwitterionic or amphoteric surfactant; and
(a3) a structuring system comprising:
(iii) from about 0.5 wt. % to about 5 wt. % of an emulsifying agent; and
(iv) from about 0.01 wt. % to about 10 wt. % of a rheology modifier;
(b) admixing the benefit phase to the cleansing phase on a flow line downstream of the batch production line, wherein the benefit phase comprises from about 0.1 wt. % to about 50 wt. % of a benefit agent; and
(c) recovering the resultant personal care composition.

2. The process of claim 1, wherein the step of forming the cleansing phase comprises the following steps, in that order:
adding water;
adding the rheology modifier under a high shear or a turbine agitation;
adding the zwitterionic or amphoteric surfactant; adding the anionic surfactant being substantially free of sulfate;
adding the emulsifying agent directly to the main mixing vessel or from a first jacketed vessel to the main mixing vessel; and
adjusting the pH such that the personal care composition has a pH of from about 4.0 to about 5.5.

3. The process of claim 1, wherein the cleansing phase is formed in the main mixing vessel including side sweep and turbine agitation, wherein the main mixing vessel includes a side sweep agitator, wherein the side sweep agitator rotates at a side weep speed ranging from about 5 to about 20 revolutions per minute; and wherein the main mixing vessel includes a turbine agitator, wherein the turbine agitator rotates at a turbine speed ranging from about 10 to about 90 rpm.

4. The process of claim 1, wherein the emulsifying agent is a glyceryl ester and/or a non-ionic emulsifier having an HLB of from about 3.4 to about 13.0;
wherein the glyceryl ester is selected from glyceryl laurate, glyceryl caprate, glyceryl caprylate, glyceryl caprylate/caprate, glyceryl stearate, and a mixture thereof;
wherein the non-ionic emulsifier having an HLB of from about 3.4 to about 13.0 comprises trideceth-3 or trideceth-4.

5. The process of claim 1, wherein the composition comprises from about 1 wt. % to about 3 wt. % of the emulsifying agent.

6. The process of claim 4, wherein the emulsifying agent is melted in the first jacketed vessel at a temperature above a melting point of the emulsifying agent.

7. The process of claim 1, wherein the rheology modifier is added to the main mixing vessel at a high shear at a turbine speed from about 35 rpm to about 45 rpm.

8. The process of claim 1, wherein the rheology modifier is chosen from sodium polyacrylate, acrylates copolymer, Acrylates/Vinyl Isodecanoate Crosspolymer, Acrylates/C10-30 Alkyl Acrylate Crosspolymer, Acrylates/C10-30 alkyl acrylate crosspolymer including stearyl side chains with less than about 1% Hydrophobic modification, Acrylates/C10-30 alkyl acrylate crosspolymer including octyl side chains with less than about 5% Hydrophobic modification, Ammonium Acryloyldimethyltaurate/Beheneth-25 Methacrylate Crosspolymer, Acrylates/Beheneth-25 Methacrylate Copolymer, Acrylates/Steareth-20 Methacrylate Copolymer, and Acrylates/Steareth-20 Methacrylate Crosspolymer, PEG-150/Decyl Alcohol/SMDI Copolymer, PEG-150/stearyl alcohol/SMDI copolymer, hydroxypropyl starch phosphate, distarch phosphate, sodium carboxymethyl starch, starch, Tapioca starch, xanthan gum, gellan gum, carboxymethyl cellulose, carboxymethyl hydroxyethyl cellulose, hydroxypropyl methyl cellulose, sodium alginate, or mixtures thereof.

9. The process of claim 1, wherein the anionic N-acyl amino acid surfactant is added relatively slowly over about 2 to about 10 minutes to the main mixing vessel at a side sweep speed from about 5 to about 10 rpm and a turbine speed from about 10 rpm to about 20 rpm.

10. The process of claim 1, wherein the anionic surfactants that are substantially free of sulfates are chosen from sodium, ammonium or potassium salts of isethionates; sodium, ammonium or potassium salts of sulfonates; sodium, ammonium or potassium salts of sulfosuccinates; sodium, ammonium or potassium salts of sulfoacetates; sodium, ammonium or potassium salts of glycinates; sodium, ammonium or potassium salts of sarcosinates; sodium, ammonium or potassium salts of glutamates; sodium, ammonium or potassium salts of lactates; sodium, ammonium or potassium salts of lactylates; sodium, ammonium or potassium salts of taurates; sodium, ammonium or potassium salts of alaninates; or mixtures thereof.

11. The process of claim 1, wherein the N-acyl alaninate surfactant is chosen from sodium cocoyl alaninate, triethylamine cocoyl alaninate, sodium lauroyl alaninate, sodium N-dodecanoyl-1-alaninate, or mixtures thereof.

12. The process of claim 11, wherein a weight ratio of sodium cocoyl alaninate to glyceryl caprylate/caprate is greater than about 5:1 to about 15:1.

13. The process of claim 1, wherein the step of forming the cleansing phase comprises the additional following steps after adding the anionic surfactants that are substantially free of sulfates, or the anionic N-acyl amino acid surfactant, in that order:
adjusting the pH; and
adding a perfume.

14. The process of claim 1, wherein the zwitterionic surfactant is chosen from cocamidopropyl betaine, coco-betaine, lauramidopropyl betaine, or mixtures thereof.

15. The process of claim 1, wherein the benefit phase is provided from a second jacketed vessel; or a jacketed vessel including a propeller turbine; and wherein the benefit agent is melted prior to admix the benefit phase to the cleansing phase on the flow line downstream of the batch production line.

16. The process of claim 1, wherein the benefit phase is admixed to the cleansing phase at a temperature above a melting point of the benefit agent.

17. The process of claim 1, wherein the benefit phase is emulsified with the cleansing phase into a mixing device at a temperature above the melting point of the benefit agent after the step of admixing the benefit phase to the cleansing phase.

18. The process of claim 1, wherein the benefit agent in the personal care composition has an average particle size from about 3 μm about 10 μm, as measured according to the Particle Size Measurement.

19. The process of claim 1, wherein the composition comprises from about 0.5 wt. % to about 15 wt. % of the benefit agent.

20. The process of claim 1, wherein the benefit agent is chosen from petrolatum, lanolin, natural waxes, synthetic waxes, volatile organosiloxanes, non-volatile organosiloxanes, lanolin oil, lanolin esters, argan oil, castor oil, soybean oil, derivatized soybean oils, maleated soybean oil, safflower oil, cotton seed oil, corn oil, walnut oil, peanut oil, olive oil, cod liver oil, sweet almond oil, almond oil, avocado oil, palm oil and sesame oil, vegetable oils, sunflower seed oil, and vegetable oil derivatives, coconut oil and derivatized coconut oil, cottonseed oil and derivatized cottonseed oil, jojoba oil, cocoa butter, shea butter, groundnut oil, camellia oil, beauty-leaf oil, rapeseed oil, coconut kernel, coriander oil, marrow oil, wheat germ oil, jojoba oil or liquid jojoba wax, linseed oil, macadamia oil, corn germ oil, hazelnut oil, walnut oil, vemonia oil, apricot kernel oil, olive oil, evening-primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil, or alternatively caprylic/capric acid triglycerides, or mixtures thereof.
